(19) United States
(12) Patent Application Publication
(10) Pub. No.: US 2003/0040002 A1
(43) Pub. Date: Feb. 27, 2003

(54) Method for providing current assessments of genetic risk

(76) Inventor: Fred David Ledley, Nedham, MA (US)

Correspondence Address: LUANN CSERR, LAW OFFICE OF LUANN CSERR, SUITE 100, 166 WHEELER AVENUE, CRANSTON, RI, 02905-2710, US

(21) Appl. No.: 10/200,978

(22) Filed: Jul. 23, 2002

Related U.S. Application Data
(60) Provisional application No. 60/310,804, filed on Aug. 8, 2001.

(51) Int. Cl. C12Q 1/68; G06F 19/00; G01N 33/48; G06F 17/60; G01N 33/50

(52) U.S. Cl. 435/6; 702/20; 705/3

(57) ABSTRACT

This invention concerns an integrated method for providing individuals with current assessments of genetic risk based on genetic tests and genomic research. While existing genetic tests can provide an estimate of genetic risk for many common diseases, genomic research is expected to provide a large number of new genetic tests that will enable more definitive assessments of an individuals risk of disease. The invention provides an integrated method and systems for providing individuals with current assessments of their genetic risk based on such advances. This method has utility in enabling individuals and healthcare professions to use genetic tests in important healthcare and lifestyle decisions.

### CLAIM OF PRIORITY

This application claims priority under 35 U.S.C. section 199(e) to U.S. provisional patent application No. 60/310,804, filed Aug. 8, 2001.

### FIELD OF THE INVENTION

This invention discloses a novel approach for using genetic tests and information from genome research together to aid individual healthcare and lifestyle decisions.

### BACKGROUND OF THE INVENTION

Currently, more than 500 different genetic tests for variations in specific genes have been described and are performed by certified laboratories. Information on tests that are commonly performed in clinical practice is available to those skilled in the art, for example through the internet location genetest.org, textbooks such as Scriver et al., The Molecular Basis of Inherited Disease, McGraw Hill, databases available through the National Center for Biological Information (NCBI) linked to the human genome project, and through MEDLINE. Establishing the clinical utility of a genetic test can require years of research and development, the genetic tests that are performed today are based on genes discovered before completion of the human genome sequence.

The completion of the human genome project has revealed a large number of genes that have not been previously characterized. While the most recent publications on the human genome sequence suggest the number may be as low as 30,000 genes, others claim to have identified over 100,000 different genes by isolating unique mRNA sequences. Either number represents a substantial increase, perhaps 10-30 fold, in the number of genes known to scientists over the number of genes that have been characterized and form the basis for existing genetic tests. Moreover, the total amount of human gene sequence that is available to scientists, including intergenic regions which are not yet identified with specific gene functions, has increased >1000 fold with completion of the human genome sequence. There has also been a dramatic increase in the number of polymorphisms or sequence variations that are known to exist among human populations. Through the work of the Single Nucleotide Polymorphism (SNP) Consortium as well as commercial and public databases, more than 2 million variances have been described, a >100 fold increase in the number that was known several years ago.

Thus, only a small fraction of genes and gene sequence variations within the human population has been studied to determine their role in health and disease. It is likely that many of these newly discovered genes and gene sequences will have an impact on human health and disease and that new genetic tests will be developed based on these discoveries. It is estimated that there may be a 10-1000 fold increase in the number of genetic tests that will have uses in healthcare and lifestyle decisions. Moreover, additional variability will be discovered in genes that are already known, adding further to the clinical value of tests that will be available. Already technologies are under development that will enable the analysis of >10-10,000 genetic tests simultaneously on a gene chip. Other genomic research is aimed at developing methods that will enable gene tests to be performed selectively on large numbers of genes at a fraction of the current cost.

### SUMMARY OF THE INVENTION

The present invention concerns methods and systems for providing individuals with current assessments of genetic risk based on genetic tests and genomic research.

The present invention describes an integrated method for providing individuals with current assessments of genetic risk for specified clinical outcomes based on genetic tests and genomic research. Specifically this invention describes methods and systems for performing assessment of genetic risk for an individual concerned about a specific clinical outcome. The invention method incorporates the following steps: (i) obtain consent from the patient for genetic testing and assessment of genetic risk for said outcome; (ii) test for genes and variations known to be involved in genetic risk for said outcome; (iii) counsel patient on test results and assessment of genetic risk; (iv) record individual's identity, consent, contact information, clinical concerns, and genetic test results in a secure and private matter; (v) monitor genomic research for genes and variations that contribute to the clinical outcome; (vi) notify the individual concerning newly discovered genes and variations that contribute to genetic risk, (vii) test for newly discovered genes and variations that contribute to genetic risk; (viii) counsel patient on test results and current assessment of genetic risk. This method, and systems for carrying out this method, can provide individuals and healthcare professions with genetic tests and tests results which can be important in making healthcare and lifestyle decisions.

The phrase “Genomic research” refers to basic and clinical research aimed at identifying the genes that comprise the human genome, their function, and their role in human health and disease. The human genome project has been undertaken with the expectation that the sequences of genes, which comprise the genome, as well as the expression of biological functions encoded by these genes, are determinants of individual development, health, and disease. Variation in gene sequences, variations in the level, location, or timing of expression of a gene, and variation in the physical, chemical, or dynamic characteristics of the products expressed from a gene are known to underlie many aspects of human individuality including physical and mental characteristics, growth, longevity, health, and disease.

An important result of genomic research is the discovery and development of genetic tests that can be used to determine how the genes of an individual predispose that individual to various health outcomes. Genomic research is commonly reported in more than 100 specialty journals in the field of genetics or genomics such as Genomics, American Journal of Human Genetics, Nature Genetics, Human Genetics, Clinical Genetics, Genetic Testing as well as general medical journals such as the Journal of the American Medical Association, New England Journal of Medicine, or Journal of Clinical Investigation and hundreds of specialty medical journals. Occasional results of particular interest to the general public are sometimes reported in general scientific journals such as Science, Nature, or Proceedings of the National Academic of Sciences. Such reports are commonly known to specialists in genetics as well as medical subspecialists who are able to use existing genetic tests in diagnosis of disorders such as cancer or neurological disease. Such reports are not commonly known to primary healthcare providers or individuals.

The term “report” refers to an article in a professional publication describing genomic research that may be useful in determining an individual's risk of a disease, disorder, or clinical outcome including, but not limited to, research and development of new genetic tests, quantitative measures of risk based on a genetic test, or uses of genetic test results to guide preventative measures or therapies.

The terms “test”, “genetic test” or “genetic testing” refer to the analysis of DNA, RNA, protein, or other biological materials in a sample from an individual to determine, without limitation, the sequence of one, or more than one, gene within the sample, the presence or absence of one, or more than one, genetic marker, variance, variation, mutation, polymorphism, or microsatellite sequence associated with a gene, the presence of one, or more than one, viral sequence, viral-like sequence, or repetitive sequence, a haplotype or genotype spanning one, or more than one, gene, the number of copies of one, or more than one, gene, the amount or characteristics of RNA or protein expressed from one, or more than one, gene, the biological function of one, or more than one, gene, the arrangement of genes within the genome, the chromosome number, or integrity of chromosomes.

One common form of a genetic test is a karyotype, in which the chromosomes of a cell from the individual are separated on a microscope slide, stained with a substance that enables the different chromosomes to be distinguished by microscopy, and then examined by an expert or by using a computer to determine the number of chromosomes and their integrity. Another common form of a genetic test involves sequencing one or more genes to determine whether the sequence of a particular gene corresponds to the sequence known to encode normal activity of the gene product, or a variant which may be correlated with abnormal function or disease. Gene sequences can be determined from gels, using automated sequencers, using gene chips, mass spectroscopy or other methods known in the art. Another common form of a genetic test involves determining how much mRNA derived from specific genes is present in a cell by hybridizing mRNA extracted from a tissue to a grid or chip that contains probes for different specific mRNAs. Other forms of genetic tests involve the identification and analysis of specific proteins by mass spectroscopy, electrophoresis, or binding to natural or synthetic substrates or antibodies. Many different biological materials may be obtained from an individual for the purposes of performing a genetic test such as, for example, blood, tissue scrapings, hair, or bodily fluids or secretions.

The present invention provides methods and systems for providing individuals with a current assessment of genetic risk based on advances in genomic research. The methods and systems of the invention can provide efficient application of these advances to individuals concerned with specific diseases, disorders, or clinical outcomes as well as individual healthcare and lifestyle decisions.

Many genes contain more than one variance. In such genes, a genetic test for any one variance within a gene sequence may not accurately reflect variations in the structure or function of the gene or its contribution on a clinical outcome. Two or more variances may have independent effects on structure, function, or expression of a gene. Often, however, multiple variances within a gene may act together in a synergistic or antagonistic manner to impart a structure, activity, pattern of expression, function, or clinical outcome that is unique to a particular combination of variances.

The terms “haplotype” or “genotype” refer to particular combinations of sequences present within a gene. Gene tests may identify single variances within the gene, variances at several positions within a gene, the sequence of a gene, or the haplotype of a gene. For example, two different variances in the apolipoprotein E (apoE) are known, and both variances must be considered together to differentiate the characteristic effects of the apoE4 genotype in cardiovascular disease or Alzheimer's disease from the effects of apoE2 or apoE3. Consideration of a single variance would fail to elucidate the involvement of the apoE gene in these disorders; therefore the tests that are performed are designed to identify both variances. With the discovery of >2,000,000 variances within the genome, it is expected that many more genes with multiple variances that affect activity will be identified. It can be recognized that as additional variances in genes are identified, it can be advantageous to perform tests for such variances in order to provide a meaningful assessment of risk.

The term “gene” is commonly known in the art and is a linear sequence of nucleotides within the human genome that encodes a biological function. A gene commonly directs the expression of RNA or protein which may be directly responsible for carrying out the function encoded by the gene, or the RNA or protein may be modified to carry out such functions. The gene may include introns, exons, promoters, or other sequences that are involved in directing the biological function. It would be recognized by the skilled artisan that the sequence of nucleotides (A, G, C, T) within the gene which encode its function may vary in different individuals, and that variances or mutations within the sequences of nucleotides may change the function. The term “mutation” refers to a specific sequence within a gene which may differ among individuals which contributes to a specific activity of a gene or gene product including, but not limited to, changes in the structure, activity, expression, availability, modification, processing, specificity, or function of the gene product.

The terms “genetic marker”, “polymorphism”, “single nucleotide polymorphism” (SNP), or “micro satellite sequence” are specific sequences within a gene that can differ among individuals and can be used to identify genes with specific functions. These terms are sometimes used to imply that the specific variation in sequence does not alter the function of the gene, nevertheless such sequences could be associated with characteristic activities of the gene or gene product. Moreover, a skilled artisan would recognize that a sequence variance that has detrimental effects in one circumstance can have beneficial effects in others, and that the distinction between a “mutation” and/or a “polymorphism” is less important than the association of a specific sequence variance with a specific clinical outcome. The skilled artisan will also recognize that the terms “variance”, “variation”, “mutation”, “genetic marker”, “polymorphism”, or “SNP”, in genome research independently can each refer to differences in gene sequences, or positions in the genome where differences in the sequence are found between different individual, and are often used interchangeably in describing a genetic test, since each term can be used in making an assessment of genetic risk.

Relationships among genes are recognized based on similarities in structure and function, activities that contribute to common biological pathways, or activities contributing to a common pathological process.

The term “gene family” refers to genes that share common structural or functional characteristics. Some genes within a gene family may commonly exhibit structural similarities due to comparable function or evolution and may contain sequence identities, common motifs, or common functional elements. It is recognized that genes within a family often carry out analogous biological functions, and that mutations in closely related genes can lead to similar clinical outcomes.

The term “pathway” refers to a sequential or intersecting set of biological functions. Many different genes may be required to carry out or support complex biological functions such as the synthesis of biological compounds, the construction of cellular or somatic structures, or the regulation of a process within the body. It is recognized that genes that contribute to a common pathway often work in a coordinated fashion, and that mutations or variations in any gene along the pathway could alter the characteristic structure, level, or expression of the end product.

Multiple genes are commonly in pathological process. Most disorders are characterized on a molecular level by changes in the level, location, or activity of many different gene products. Such genes may have synergistic or antagonistic actions in the pathological process, and each gene may contribute to the risk of the clinical outcome. For example, many different products comprise the pathological findings of the brain in Alzheimer's Disease, plaques in cardiovascular disease, the inflammatory lesion of arthritis, or the malignant cell in cancer. With the discovery of 30,000-100,000 new genes through genomics, many more such associations will be recognized, adding to the complexity of genetic testing and assessments of genetic risk for specific clinical outcomes. A skilled artisan would recognize that as such genes and associations are identified, it can be advantageous to perform tests for several genes within a family, on a pathway, or involved in a pathological process to identify risk factors for a specific clinical outcome in order to provide a meaningful assessment of risk.

The invention describes an integrated method for providing individuals with current assessments of genetic risk for a specific clinical outcome based on genetic tests and genomic research. The invention describes methods, systems, and internet/network sites for performing a current assessment of genetic risk in individuals concerned about a specific clinical outcome through integrating genetic tests for genes and variations with systems that provide for retesting for newly discovered variances and genes and providing recounseling based on the results of such tests as well as providing an individual current knowledge of the association of a gene test result with a clinical outcome reported from genomic research.

The invention provides genetic tests useful in the field of medicine for predicting a clinical outcome, diagnosing a genetic disease or disorder, determining an individual's propensity to multifactorial diseases or disorders, and predicting an individual's response to therapeutic drugs.

The terms “disease” and “disorder” are often used interchangeably and refer to recognized morbid or pathological events and are commonly catalogued in textbooks of medicine and standard classifications of disease such as the International Classification of Disease (ICD).

The term “clinical outcome” refers to any observable clinical event such as, for example to, health, morbidity, or mortality; growth, development, aging or longevity; the onset, progression, course, remission, relapse, symptoms, signs, or pathology of a disease or disorder; cognitive functions, behaviors, psychoses, or dementia; as well as drug response, or drug toxicity or the response to any intervention involving drugs, nutrition, lifestyle change, education, or surgery as well as the application of non-allopathic therapies such as traditional or folk medicines, osteopathy, or chiropractic medicine.

The invention describes an integrated method and sysgtems for providing individuals with current assessments of genetic risk for a clinical outcomes based on genetic tests and genomic research. The method can provide individuals and healthcare professionals with genetic tests and genetic test results for use in making decisions concerning healthcare and lifestyle in relation to concerns about specific clinical outcomes.

The invention also provides an integrated method and systems for providing individuals with current assessments of genetic risk for a specific clinical outcome based on genetic tests and genomic research, in which the method can extend the capabilities of the healthcare provider in providing genetic counseling to an individual. The invention describes methods, systems, and Internet sites for performing current assessments of genetic risk in individuals concerned about a specific clinical outcome. The invention provides for retesting and recounseling based on reports from genomic research and development of additional genetic tests useful in an assessing the genetic risk of a specific clinical outcome. This invention specifically provides for a current assessment of risk by integrating a record with information concerning the individual, their family history, genetic test results and other factors which may contribute to their risk of one or more clinical outcomes with monitoring for reports of new information concerning genes, variations, genetic tests, analytical methods, or calculations that are relvant to an assessment of the genetic risk of such outcomes. These systems are preferably integrated with the ability to notify the individual whan new information is reported that could be used to make a current assessment of risk. Perferably systems that integrate the report, monitoring, and notification are integrated also with appropriate informed consent, testing, counseling, retesting, and recounseling to provide a current assessment of risk.

The invention more specifically comprises an integrated method for providing individuals with current assessments of genetic risk based on genetic tests and genomic research that specifically addresses problems that presently limit genetic testing in clinical practice. The invention includes methods for performing a current analysis of genetic risk in an individual by identifying the individual's concern(s) about a specific clinical outcome, for example, by taking and/or cataloging a family history, and providing the individual with genetics tests and counseling based on current reports of new genes and variations that can contribute to risk related to the clinical outcome. The integrated methods, systems, and sites of the present invention allow individuals and healthcare professionals the use of current information concerning genetic tests and genetic test results in making healthcare and lifestyle decisions. Thus, the invention enables current assessment of genetic risk in individuals concerned about a specific clinical outcome by integrating, without limitation, the following steps: (i) obtain patient consent for genetic testing and assessment of genetic risk for said outcome; (ii) test for genes and variations known to be involved in genetic risk for said outcome; (iii) counsel patient on test results and assessment of genetic risk; (iv) record individual's identity, consent, contact information, clinical concerns, and genetic test results in a secure and private matter; (v) monitor genomic research for genes and variations that contribute to said clinical outcome; (vii) notify the individual concerning newly discovered genes and variations that contribute to genetic risk; (vii) test for newly discovered genes and variations that contribute to genetic risk; and (viii) counsel patient on test results and current assessment of genetic risk.

The term “individual” refers to, any person including a patient as well as family, friends, or agents of a person or patient other than those working in their capacity as health care providers.

The term “health care provider” or “provider” are commonly known in the art and includes, without limitation, physicians, practitioners specialized in genetics such as M.D. or Ph.D. trained geneticists or genetic counselors, practitioners specializing in the care of individuals with disabilities or inherited genetic diseases.

The invention comprises an integrated method for providing individuals with current assessments of genetic risk for a clinical outcome based on current reports of genomic research. The invention includes integrated methods for performing a current assessment of genetic risk for individuals concerned about a specific clinical outcome by testing for genes and variations known, on the basis of current reports, to be involved in genetic risk for a clinical outcome and further providing counseling to the individual patient and/or regarding the test results and further providing can assessment of genetic risk based on current knowledge. This invention extends current practice by establishing a record of the individuals concern regarding a specific clinical outcome, monitoring genomic research for genes and variations that contribute to said clinical outcome, notifying the individual of newly discovered genes and variations that contribute to genetic risk, and then offering the individual genetic and/or clinical tests, as described herein for such genes and provide counseling concerning the test results in order to provide a current assessment of the individual and/or patient genetic risk in return to the test findings.

The term “record” or “recording” refers to a system containing information including, but not limited to, an individual's identity, informed consent provided for genetic testing, contact information which would allow notification of an individual when opportunities for retesting and recounseling for a current assessment of risk are identified, the individuals concerns about specific clinical outcomes, and the results of previous genetic tests. The record can also contain information about personal and developmental history, family history, clinical laboratory data, images, findings on physical exam, and previous illnesses and therapies that can be useful in determining a genetic test result. The invention comprises system for establishing, maintaining, or accessing such a records that is integrated with systems for monitoring and notifying, so that genomic research may be effectively monitored for genes and variations that contribute to the outcome of concern to the individual as noted in said record, and the individual can be notified by using the contact information contained in the record(s) when such genes or variations or additional information about genes or variations, are identified. Contact information can include, for example, an individual's address, telephone number, fax, or email, comparable information about a healthcare provider, relative, or other third party sufficient to enable notification or may comprise identifying information and a password that an individual can provide when entering a specific web site. Methods for establishing and maintaining individual medical records are known in the art and can involve storing information in different media or at different locations to protect privacy and security. The maintenance of medical records and the use and protection of personal medical information is regulated by HIPA (Health Insurance Portability Act) and by other state and federal regulations.

The term “access” refers to the ability of an individual, provider, or system to retrieve, receive, or review the information in specific record.

In a specific embodiment, the invention provides methods and systems for providing an individual with a current assessment of their genetic risk for a clinical outcome while protecting the individual's privacy and confidentiality and adhering to regulatory guidelines concerning informed consent, genetic testing, and genetic counseling. This is achieved using systems through the use of systems that monitor and perform an assessment of risk for individual records. Specifically, the system may, on a regular basis, for example, daily, weekly, biweekly, or monthly compose an index of new information obtained through monitoring. Individual records are preferably accessed on a confidential basis sequentially and independently, and specific clinical outcomes that are addressed in the record can be correlated with the availability of new information in the index. Individual records can be surveyed on a daily, weekly, biweekly, or monthly basis or upon a prompt when an individual accesses the system through the internet, or alternatively, by telephone, mail or other medium. Alternatively, the system could be designed to keep an index of individuals concerned about a specific health outcome, and any individuals included in the index could be notified, or testing performed for that individual in accordance with the informed consent, when monitoring identifies new information for a current assessment of risk. Those skilled in the art know that automated systems such as the internet and computer software are inherently more efficient for correlating new information identified through monitoring but that such an index comprises a measurable risk to privacy. The risk to privacy is minimized by further encoding the identity of the individual within the index.

The term “system” is known in the art and refers to both to interacting or interdependent components and also to an organized, coordinated procedure for achieving a specific purpose. Examples of systems include both computer programs as well as organizations of individuals or individuals and computers that operate in an organized or coordinated manner, for example, in accordance with standard operating procedure. A system or process can commonly be described through flow diagrams or organizational charts and commonly incorporate standard procedures that describe each of the steps required to achieve a specific purpose. A system can be comprised of automated components which can include, hardware and software, communications equipment, links to the Internet, Internet connections, a site/location on the Internet, or methods for automatic mailing as well as non-automated components which are commonly performed using standard procedures or standard operating procedures.

A “standard operating procedure” is a document that describes the agreed and validated procedures for carrying out process and may constitute a component of a system. Standard operating procedures can provide for a series of actions each of which can lead to specific results as well as a set of contingencies for subsequent actions based on such results. A system can comprise a self contained set of components automated and non-automated components working in an integrated fashion and can also involve integration of systems that are internal to a specific organization or group of providers, and those that are external.

The invention provides a system designed to carry out two or more of the steps of obtaining the consent of an individual and/or patient for genetic testing and assessment of genetic risk for a clinical outcome, testing for genes and variations known to be involved in genetic risk for said outcome, counseling the individual on test results and assessment of genetic risk, recording the individual's identity, consent, contact information, clinical concerns, and genetic test results in a secure and private matter, monitoring genomic research for genes and variations that contribute to said clinical outcome, notifying the individual concerning newly discovered genes and variations that contribute to genetic risk, retesting for newly discovered genes and variations that contribute to genetic risk, and counseling the individual and/or patient on test results and current assessment of genetic risk. Components of this system can include, computer hardware, software, professional services, interactive devises, laboratory equipment for genetic tests, Laboratory Information Systems, the Internet, sites on the Internet, equipment for automatic mail or fax, written materials, standard operating procedures, publications in scientific journals, databases, data retrieval software, written documents, documents transmitted by email, fax, or mail, and information transmitted by oral communication or telephone.

The term “integrated” as used herein available through a linked system or systems. The present invention describes systems with utility for integrating the steps necessary to provide a current assessment of genetic risk. A specific feature of this invention concerns a site assessable via the Internet that integrates the steps necessary to provide a current assessment of genetic risk with utility in providing a current assessment of genetic risk to individuals, recognizing that some of these steps may not take place over the Internet but may involve alternative media including, for example mail, fax, interactive television, telephone, or publication.

Specific elements of this invention are computer software and hardware capable of carrying out the unique methods and embodiments described including the concept, design, construction, appearance, organization, function, and content of a web or Internet location site that integrate the multiple steps required for providing genetic services. Specific embodiments also include software and hardware capable of carrying out the unique methods and embodiments described herein including without limitation, the concept, design, construction, appearance, organization, function, and content of a web or Internet location site integrated with alternate media including for example mail, fax, television transmission, interactive television transmission, or telephone.

Another element of the present invention is an informed consent document and process that provides both for a genetic test such as a genetic test on a selected gene or variances in said gene known to comprise risk factors for a specific clinical outcome, and for future genetic tests such as tests on additional variances on said gene that may be reported through genomic research. The informed consent can enable testing for newly discovered variances in selected genes, additional genes within a gene family, additional genes with related functions, additional genes on a pathway, additional genes involved in a pathological process, or additional genes discovered to contribute to the genetic risk of a clinical outcome. This informed consent portion can provide for tests as described herein to be performed without additional notification of the individual or healthcare provider, with the test results provided either to the individual or to a designated healthcare provider. Alternatively, the informed consent may provide for notification of the individual by for example, posting information on a site on the Internet, by email, mail, telephone, fax, oral communication, or other media known in the art. Alternatively, the informed consent portion can provide for notification of the individual and the opportunity to assent to such genetic test being performed by the individual responding through email, mail, telephone, fax, oral communication, or other media known in the art. In all cases, the informed consent protein will be prepared in a manner that is in strict accordance with laws governing the informed consent process and will inform the individual of the risks of genetic tests and provide for counseling to assist the individual in determining their genetic risk and selecting appropriate healthcare or lifestyle interventions.

The invention comprises an integrated method and systems for performing a current assessment of genetic risk in individual concerned about a specific clinical outcome incorporating two or more than two the following steps: (i) obtain consent of the patient for genetic testing and assessment of genetic risk for said outcome; (ii) test for genes and variations known to be involved in genetic risk for said outcome; (iii) counsel the patient on test results and assessment of genetic risk; (iv) record the individual's identity, consent, contact information, clinical concerns, and genetic test results in a secure and private matter; (v) monitor genomic research for genes and variations that contribute to said clinical outcome; (vi) notify the individual concerning newly discovered genes and variations that contribute to genetic risk; (vii) test for newly discovered genes and variations that contribute to genetic risk; and (viii) counsel patient on test results and current assessment of genetic risk.

The invention provides an individual with a risk assessment, based on advances in genomic research including the discovery and development of new genetic tests for new variances or new genes or changes in the calculated risk based on the continuing accumulation of clinical data from clinical trials, clinical research, and clinical practice.

The term “contract” is commonly known in the art and refers to a binding agreement between two or more parties. In the present invention, a contract is established in which an individual pays for a current assessment of risk; the assessment of risk is paid for by a third party. One aspect of the present invention is a contract in which an individual pays initially for an ongoing assessment of risk using the systems of this invention. The contract can require payments for individual elements of these systems, for example, for each genetic test performed, for maintaining a record, or for each counseling session.

The invention can be used for any disease, disorder, or clinical outcome known in the art for which genomic research has identified and can identify genetic tests that constitute risk factors. A skilled artisan would recognize that the present invention is not limited to the type or number of diseases that can be attributed to genetic origins. Such diseases, disorders, or clinical outcomes can be found in textbooks of medicine, surgery, or medical subspecialties, in classifications of disease such as ICD, textbooks of genetics, and catalogues of such information such as Mendelian Inheritance in Man. The invention is useful for common diseases that are generally considered to be multifactorial or polyeni in origin including, but not limited to, heart disease, hypertension, heart failure, coronary vascular disease, cerebral vascular disease, stroke, peripheral vascular disease, arthritis, rheumatoid arthritis, Lupus Erythematosis (SLE), psoriasis, asthma, reactive airway disease, COPD, osteoarthritis, osteoporosis, hearing loss, cataracts, renal failure, nephritis, hepatic failure, hepatitis, pancreatitis, diabetes, infection, cancer, drug toxicity, drug resistance, drug dependence, neurological diseases, dementia, Alzheimer's disease, psychosis, neuroses, metabolic diseases. The invention may also be used for monogenic disorders where retesting may identify genes that affect the expressivity or penetrance of the mutant disorder in different individuals.

### DETAILED DESCRIPTION OF THE PREFERRED EMBODIMENTS

Genetic tests are expected to have an increasingly important role in healthcare management, enabling predisposition testing and interventions to prevent disease before morbidity is apparent, providing early diagnosis and therapy, and optimizing pharmacological interventions with drugs that are likely to be safe and effective for an individual. To date, genetic tests have also been developed for genes that predispose an individual to diseases including, for example, atherosclerosis, heart failure, stroke, anemia, cancer, clotting disorders, dementia, endocrine diseases, and pulmonary diseases. The terms “predisposition” and “risk” both refer the likelihood that an individual will exhibit a specific clinical outcome.

The term “genetic test” as used in this invention includes tests which determine the structure, characteristics, amount, or activity of certain chemical entities including, for example, proteins, protein derivatives such as glycoproteins, lipoproteins, or phosphoproteins, lipids, carbohydrates, small-molecule organic compounds, and inorganic compounds measured in tissue or body fluids. The structure, amount, or activity of such chemical entities often reflects the structure or activity of one or more genes, and the results of such tests often enable direct inferences to be made concerning the structure or activity of one or more genes. For example, electrophoresis of hemoglobin extracted from red blood cells can reveal a charge of the hemoglobin molecule caused by the sickle cell mutation in the hemoglobin gene; an increase in the amount of phenylalanine in the blood or urine can reveal the presence of mutations in the gene for phenylalanine hydroxylase; the concentration of specific salts in the urine can reveal the presence of mutations in the gene for 21-hydroxylase, the concentration of salts in sweat can reveal the presence of mutations in the CFTR gene, the ability of a specific monoclonal antibody to hybridize to a tumor cell may determine whether a chromosomal rearrangement has taken place between the her-2 and neu genes in that cell; and a measure of Thiopurine Methyltransferase enzyme activity may reveal the presence of mutations in the TPMT gene. One skilled in the art will recognize that it is frequently more convenient and less expensive to measure the structure or activity of a protein with such methods as electrophoresis or antibodies or to measure metabolites in blood or urine than to perform an analysis directly on DNA or RNA with current technologies. Such tests can be used interchangeably with tests that directly analyze DNA or DNA in the present invention.

Many different types of tests can reveal information about variations in gene sequences, expression or function including, but not limited to, tests for proteins in serum, blood cells, tissue sample, assays for the expression of specific genes measured at the RNA or protein level, and assays for metabolites that are characteristic of specific gene dysfunctions.

An exemplary purpose for performing a genetic test on an individual is to establish an association between the sequence of one, or more than one, gene within the sample, the presence or absence of one, or more than one, genetic marker, variance, variation, mutation, polymorphism, or microsatellite sequence associated with a gene, the presence of one, or more than one, viral sequence, viral-like sequence, or repetitive sequence, a haplotype or genotype spanning one, or more than one, gene, the number of copies of one, or more than one, gene, the amount or characteristics of RNA or protein expressed from one, or more than one, gene, the biological function of one, or more than one, gene, the arrangement of genes within the genome, the chromosome number, or integrity of chromosomes and a specific clinical outcome. Such associations are generally established through clinical trials. It is often necessary to perform multiple clinical trials to achieve a consensus on the contribution of a particular finding towards an individual's genetic risk of a specific clinical outcome or even to perform meta-analyses that combine the data from several different trials. Genetic tests may initially be made available to individuals based on reports on small populations that demonstrate the utility of the test. Such studies are commonly supplemented by reports on larger numbers of patients as the tests enter clinical practice, and these larger studies often provide more accurate data on the association between the test results and a specific clinical outcome. It is recognized that as clinical trials are completed, it can be advantageous to obtain and provide such information to individuals to provide a current assessment of risk. Frequently there will be hundreds of independent reports concerning the effect of a specific gene or a specific variance on the risk of a specific outcome. Such reports can be amalgamated through the use of review articles, meta-analysis or through the issuance of consensus guidelines or recommendations concerning the assessment of risk based on a specific gene or variance. Such reviews, analysis, guidelines, or recommendations themselves comprise reports.

A “test result” or “genetic test result” comprises information concerning the sequence of one, or more than one, gene within the sample, the presence or absence of one, or more than one, genetic marker, variance, variation, mutation, polymorphism, or microsatellite sequence associated with a gene, the presence of one, or more than one, viral sequence, viral-like sequence, or repetitive sequence, a haplotype or genotype spanning one, or more than one, gene, the number of copies of one, or more than one, gene, the amount or characteristics of RNA or protein expressed from one, or more than one, gene, the biological function of one, or more than one, gene, the arrangement of genes within the genome, the chromosome number, or integrity of chromosomes together with information on how such findings are associated with a specific clinical outcome. Genetic test results are used in conjunction with information from clinical trials which provide quantitative information on the association of a specific test result with a clinical outcome to provide an assessment of genetic risk.

“Genetic counseling” or “counseling” means providing information to an individual concerning the interpretation and use of a genetic test or genetic test result. Counseling is considered to be an essential step in providing an individual with an accurate assessment of their genetic risk and providing an individual with assistance in the use of this information in making decisions regarding healthcare, lifestyle, family planning, or other activities. Counseling is generally performed by a healthcare provider who has specialized training in genetics and is trained in how to interpret the results of a genetic test and provide genetic counseling including physicians, Ph.D. geneticists, or individuals with a specialized Masters or Doctorate level degree in genetic counseling. Counseling generally occurs in the office of a healthcare provider and commonly consists of a single session with a provider. For certain tests, counseling may involve two sessions, one before the test to counsel concerning informed consent, the second after the test to counsel concerning the genetic test results. The process of genetic counseling is described in many articles and textbooks. The process commonly includes obtaining a family or medical history from an individual, discussing the benefits and risks of a genetic test, communicating the results of a genetic test, and explaining the medical significance of the genetic test results, elaborating on various health related choices the individual may make on the basis of the genetic test results, and discussing the consequences of genetic test results to others in the extended family or to the individuals designated physician.

Generally counseling is performed in an office or outpatient setting and is billed on a fee for service basis. Counseling can be provided by providers who are not specially trained in genetics. Subspecialty physicians can have more expertise in the interpretation of genetic tests in specific fields, however, few have training in genetic counseling. Subspecialists commonly use genetic tests for diagnosis rather than for risk assessment since they are not often involved in patient care before the onset of disease or abnormal condition. For example, while oncologists commonly have considerable experience regarding the genetic risk of cancer, few individuals seek oncologists until a diagnosis of cancer is suspected. Similarly, neurologists have considerable experience regarding the genetic risk of dementia, but individuals are notoriously reluctant to seek a neurologist until a diagnosis of dementia is suspected. Subspecialists may refer individuals to professionals trained in genetics for counseling related to the potential effect of a genetic variation on the individual, their progeny and other family members.

Information about genetic risk is available from sources other than healthcare providers. Patient support groups specializing in certain disorders or classes of disorders are often an important source of information for individuals. General information is also available on the Internet or World Wide Web, for example at Internet locations such as genetests.org, geneclinics.org, ncbi.nih.gov, rarediseases.org, or genesage.com. The information locations such as these are provided in a very straightforward and scientific manner. The present invention offers a method to integrate such information with consent, testing, counseling, records, monitoring, retesting, and recounseling as described herein.

Genetic tests can be performed for the purpose of diagnosis and/or for the purposes of determining an individual's genetic risk. Examples of genetic tests performed for diagnosis can include: the use of a sweat chloride test or molecular analysis of the CFTR gene in a child with failure to thrive and recurrent pulmonary infections to determine whether the child has cystic fibrosis; a chromosome test performed for an individual with heart disease and dysmorphic features to determine whether the individual has Downs syndrome; an analysis of the CMT gene locus performed for an individual with neurological symptoms to determine if that individual has Charcot Marie Tooth Disease; and analysis of serum proteins in an individual with emphysema to determine whether the individual has alpha(1) antitrypsin deficiency.

An important application of genetic testing is the diagnosis of “single gene” or “monogenetic” disorders, meaning that the risk of the disease is predominantly due to mutations within a single gene. Such disorders are generally rare. Some common examples of monogenic disorders are Cystic fibrosis due to monogenetic mutations in the CFTR gene, phenylketonuria due to mutations in the phenylalanine hydroxylase gene, and sickle cell disease due to mutations in the B-globin gene. Such disorders are characterized by classical patterns of Mendelian inheritance such as recessive, dominant, or X-linked inheritance. While monogenic diseases can exhibit variable penetrance or expression due to environmental factors or the effects of other genes, an individual is commonly considered to have the disease, or at least a subclinical form of the disease, if they inherit one or more mutations in the causative gene. It should be noted that a monogenetic disorder can be characteristically associated with a specific variance within a gene, or may arise from many different variances occurring at various locations within that gene. For example, genetic tests for sickle cell disease identify a single base change that causes sickle cell disease. In contrast, the state-of-the-art testing for cystic fibrosis may identify more than 80 different variances in CFTR, each of which is known to interfere with the normal activity of this gene or its gene product, thus causing cystic fibrosis. It would be recognized by one of skill in the art that it would be advantageous to test for as many known variations as possible to provide the best possible assessment of risk.

Most common healthcare problems including, for example, cardiovascular disease, cancer, and dementia are considered to be “multifactorial” or “polygenic”, meaning that they are caused by a combination of multiple factors including variances in one or more genes as well as environmental factors and temporal factors associated with aging. For example, mutations in apoE, the Low Density Lipoprotein (LDL)-receptor, Lipoprotein lipase, angiotensinogen, or methylenetetrahydrofolate reductase (MTHFR) each contribute to cardiovascular disease, a person's diet, drugs, and lifestyle also have an impact on the onset of such a disease. BRCA-1, BRCA-2, P53, her-2, and new can each contribute to breast cancer, though environmental factors such as carcinogens, the history of pregnancies, and the administration of hormones also have an impact. Multiple genes including apoE, presenillin, and antichymotripsin have been shown to contribute to the risk of Alzheimer's Disease.

Rarely, severe mutations in one or more of these genes may be sufficient to cause a disease. For example, severe defects in the LDL-receptor or MTHFR cause recognizable monogenic disorders. However, most common variances in these genes are not associated with discrete clinical outcomes. Most importantly, an individual is generally not considered to have cardiovascular disease just because they inherit a common, pathogenic variation within a gene such as apoE, the LDL-receptor, Lipoprotein lipase, angiotensinogen, or methylenetetrahydrofolate reductase, nor is an individual considered to have cancer or dementia if they inherit genes or mutations that contribute to these disorders. Rather, mutations in such genes are understood to be risk factors that make an individual more susceptible, or predisposed, to disease in the presence of other events.

Examples of genetic tests performed for determining an individual's genetic risk can include: molecular analysis of the apoE gene, the MTHFR gene, or the angiotensinogen gene to determine the presence of mutations known to predispose to atherosclerosis; the molecular analysis of the BRCA(1) or BRCA(2) gene in an individual with a family history of breast cancer to determine the presence of mutations known to predispose one to cancer, the analysis of the IL-1 gene to determine the presence of mutations known to predispose a person to peridontal disease. Many other genes which can be used to determine an individuals risk of a clinical outcome are known in the art and are found in references such as genetests.org, Scriver et al., The Molecular Basis of Inherited Disease, or textbooks of medicine or genetics.

The most important advances in medicine arising from genomic research are likely to be those that enable more accurate prediction of an individual's risk of common disorders so that changes in healthcare or lifestyle can be implemented to prevent or modify the clinical outcome. Multifactorial disease, in which both genetic and environmental factors can contribute to the pathogenesis of a disease or its prevention, are preferred targets for the development of genetic tests, since changes in lifestyle or healthcare which comprise changes in the environment may be expected to have an impact in preventing or treating such diseases.

An individual's risk of most common such as for example, cancer, heart disease, stroke, osteoporosis, arthritis, dementia, and others is generally believed to relate to one, or more than one, variation, occurring in multiple genes. For example, an individual who inherits variances in two different genes such as BRCA as well as p53, each of which independently increases the risk of cancer, can have a substantially higher risk of cancer than an individual who inherits variances in only one gene. Multiple variances can act in a synergistic manner to increase the likelihood that an individual will suffer a particular disorder or to increase the severity or rate of progression of the disorder. Sometimes variances may have opposing actions. Some variances are known to be protective, so that an individual's risk of disease arising from a variance in one gene may be reduced by protective variances within the same gene or within other genes. Because of the multifactorial, polygenic nature of most common diseases, a complete assessment of risk will commonly require analysis of more than one gene and frequently multiple variances. Since only a small fraction of the genes and variances that contribute to common disease have been elucidated to date, any assessment of risk is necessarily incomplete. This invention provides a method and systems for providing individuals with a current assessment of risk as new information is reported.

The terms “genetic risk” or “assessment of risk” refer to a quantitative and/or statistical measure of the likelihood that an individual will have a certain detectable and/or observable clinical outcome as a result of variations in one or more than one gene. The risk is generally expressed as the fold increase in risk of a particular clinical outcome, the likelihood that an individual will experience a particular clinical outcome if they have a specific genetic variation, or an odds ratio. Methods for making an assessment of risk are known by those skilled in the art as commonly described in medical journals in conjunction with the discovery of genes that are considered risk factors for specific disease.

A gene is considered to be a “risk factor” for a specific clinical outcome, condition and/or disease if a specific variation in that gene is associated with a higher frequency of that outcome. The terms “susceptibility”, “predisposition”, and “risk” are sometimes used interchangeably to describe the likelihood of an individual exhibiting a disease, disorder, or clinical outcome.

An important goal of genomics research is to be able to determine an individual's genetic risk for a specific clinical outcome by identifying one or more variations in one or more genes that can contribute to that disorder or clinical outcome. The identification of genetic risk factors before the onset of disease will enable the implementation of medical or lifestyle interventions that may be effective in preventing the disease. For example, individuals with mutations in apoE are particularly at risk for cardiovascular disease resulting from elevations in cholesterol that can be prevented through diet and appropriate pharmacological therapy. In contrast individuals with mutations in angiotensinogen are at particular risk for cardiovascular disease that may respond to salt restriction and diuretics, and individuals with mutations in MTHFR are at particular risk for cardiovascular disease due to elevations in homocysteine that may be prevented with high dose vitamin therapy.

In determining an individual's genetic risk of a disorder or clinical outcome, it is important to take into consideration multiple variations that may occur in a gene, as well as the presence of variations in different genes comprising a gene family or contributing to a pathway or pathological process. While the identification of one mutation in one gene may indicate that an individual might be at higher risk of disease, a more accurate assessment of the degree of genetic risk and determination of the appropriate medical response must take into account all of the variations in each of the genes known to contribute to that risk. When all of genes that can contribute to a clinical outcome are not known, and when the clinical significance of gene sequence variations within such genes are not understood, it is not possible to make a definitive assessment of genetic risk. This is true for most genetic tests currently used to make an assessment of risk. As the number of genes that are known within the human genome increases as a result of genomic research, and as further genomic research ascribes specific functions to these genes in health and disease and identifies variances within these genes that change the structure, activity, expression, function or clinical outcomes associated with these genes, it will be possible to make more accurate assessments of genetic risk.

A problem inherent in incomplete knowledge of genes and variations involved in disease was identified as a major limitation in genetic testing in a recent article in The New England Journal of Medicine. The authors wrote:

“These problems can be broadly divided into psychosocial or technical in nature. From the societal view, issues related to insurance, employment discrimination, and privacy have garnered much concern and attention. Additional ethical concerns arise when no effective intervention is available and when prenatal testing is considered for diseases with late onset or minimal effects. The technical challenges associated with genetic testing can be just as formidable and are often overlooked. For example, in many diseases, not all of the genes capable of causing or contributing to pathogenesis are known. Moreover, even when the mutated gene is known, routine genetic testing may fail to identify mutations in 25 to 75% or more of the cases. As a result of these uncertainties, genetic testing that fails to find a mutation is often inconclusive. Studies have shown that these inconclusive results may be misinterpreted by the patient and physicians and are a source of great anxiety.”

The term “current assessment of risk” refers to an assessment of an individual's risk of a specific clinical outcome based on current information concerning genes and variations that are associated with that clinical outcome. It is recognized by those skilled in the art that, while an assessment of an individuals' risk of a clinical outcome may be made based on a family history and specific genetic tests, the discovery of new genes or variations involved in that clinical outcome, or additional data on the quantitative impact of a specific gene or variation on the risk of that clinical outcome may change that assessment. Specifically, with rapid progress in reports of genomic research and clinical research aimed at understanding the role of specific genes and variations in disease, an assessment of an individual's risk made at a particular point in time, based on knowledge available to those skilled in the art at that time, may not be current once additional risk factors are identified or the quantitative impact of specific risk factors is refined based on clinical research. A current assessment of risk is an assessment based on reports, genetic tests, analytical and quantitative available to those skilled in the art at a particular time. Specifically, an assessment of risk is considered to be current if it is based on information that is available to those skilled in the art monitored continuously, daily, weekly, every two weeks, three weeks and/or monthly.

In current practice, genetic testing is initiated by health care providers such as physicians, practitioners specialized in genetics such as M.D., Ph.D., or trained geneticists or genetic counselors, and practitioners specializing in the care of individuals with specific disorders, disabilities or inherited genetic diseases. A major limitation of current practice is that many healthcare providers, particularly primary healthcare providers who are most likely to assist an individual to assess their predisposition to, or risk of, disease later in life, are not familiar with the application of many genetic tests and are not current with advances in genomic research on a day to day basis. Thus, many individuals do not have current access to genetic tests and test results that may be developed through genomic research. Genetic tests are performed and genetic counseling is provided through a referral to a healthcare provider specially trained specially in genetics. The genetics professional will meet with the individual one or two times, and the responsibility for ongoing medical care will continue to reside with the referring provider. The specialty of genetics rarely provides ongoing medical care except for certain monogenetic disorders, particularly inborn errors of metabolism.

Samples are typically obtained by the health care provider, a central blood drawing service of a hospital or health care clinic, or a satellite facility of a diagnostic testing service and are commonly sent to genetic testing services (often referred to as “reference laboratories”), such as Genzyme Genetics (genzyme.com), Quest Diagnostics (questdiagnostic.com), Gene Screen (genescreen.com), or to certain clinical laboratories or hospital based, or academic research laboratories for genetic tests. Such laboratories are commonly regulated by Clinical Laboratory Improvement Act (CLIA) which sets standards for the performance and reporting of test results. The results of a genetic test are reported to an health care provider who is expected to communicate the results to the individual and provide whatever genetic counseling is necessary to allow the individual to make necessary healthcare or lifestyle decisions based on the test.

The use of genetic tests to assess genetic risk is most valuable before the onset of a disease so that measures can be taken to prevent that disease. Thus, genetic testing would be most useful in young adults. Another important problem in the use of genetic tests for genetic risk assessment factor is that young adults, for example individuals between the age of 15-45, who are most likely to benefit from genetic testing, infrequently visit healthcare providers and often have no primary providers. For example, recent data from the CDC demonstrates that ˜25% of adults age 18-20 and ˜20% of adults age 21-44 have no “usual source of health care”. Data also demonstrate that men between the ages of 15-44 average <1 visit to a healthcare professional/year, while women average <2. Thus, genetic testing and/or counseling facilities are generally unable to provide individuals with a current assessment of risk even if they are familiar with reports from genomic research of new variances, new genes, or new data on the impact of specific variances of genes is reported from genomic research.

The irregular utilization of healthcare by young and healthy adults and lack of continuity is a particularly difficult problem for the effective application of genetic tests to assess genetic risk. Most individuals have many different healthcare providers during their lives. Pediatricians commonly will care for individuals only through the age of 18 or sometimes through college. It is common for individuals to have different healthcare providers and different payers during their lives. Current data indicates that individuals only retain healthcare plans for an average of 2-3 years before moving to different plans because of different employment, choices of plans, or changing medical needs. Moreover, individuals will often use different providers simultaneously for different healthcare concerns. For example, women will commonly use an OB/GYN and may receive primary medical care elsewhere. Often there is little communication between these different professionals and little integration of medical records or information. Thus, information about a genetic test or test result that is obtained by one healthcare provider may not be available to other providers. If different genetic tests are performed by different healthcare providers and these records are not combined, then any assessment of genetic risk based on one test or the other (but not both) will be incomplete. The present invention describes an integrated method for providing individuals with current assessments of genetic risk for a specific clinical outcome based on genetic tests and genomic research including a consolidated record of genetic tests and tests results. The invention and methods allow for a comprehensive and current and updateable assessment of risk that is not possible with current healthcare practices.

There is profound concern about the potential misuse of genetic information to discriminate against individuals who may have specific genetic variances. There is particular concern that individuals with specific genes or variant forms of genes may be discriminated in terms of access to health care, the cost of health care, employment, insurance (life, disability, health, etc.), and in social interactions. The legacy of eugenics, persistent racism, and popular perceptions concerning genetic and ethnic differences among individuals heightens concern that genetic information about individuals will be used for discrimination. There is extensive literature on the importance of maintaining the privacy and confidentiality of genetic records to prevent such abuse, and laws designed to ensure the privacy of genetic records and prohibit discrimination are now widespread. Nevertheless, individual concern that the results of genetic tests may be misused by health care providers, insurers, employers, or even the government continues to limit the utilization of many genetic tests. One of the limitations of current practice is that privacy assurance for individuals who may be concerned that results of genetic tests could be used to discriminate against them, and assurances that risks of confidentiality due to the number of different people and services that are involved are not adequate. Every interaction with a different health care provider and every medical record that contains information on genetic tests and the results of genetic tests is a potential risk to an individual's privacy. This risk is exacerbated if it is necessary to test for different genes at different times during a person's life through different healthcare networks.

The invention includes methods for integrating and providing individuals with current assessments of genetic risk for a specific clinical outcome based on genetic tests and genomic research in which the individual can be empowered to control and consolidate information about their genetic risk and in which such information can be made available selectively to healthcare providers designated by the individual and who need to know such information in order to deliver appropriate healthcare to the individual.

Perhaps the most difficult problem in genetics is the question of when is it appropriate to begin offering genetic tests and providing individuals with an assessment of genetic risk. It is frequently argued that it is not appropriate or ethical to offer an assessment of genetic risk based on current knowledge, knowing that a large number of genetic tests, which will allow a more accurate assessment of risk, will be developed over the next 5-10 years. However, denying individuals information that is currently available can deprive many of the chance to implement changes in healthcare or lifestyle which could be effective in preventing or modifying the course of a specific disease or clinical outcome.

An important aspect of genetic testing is the importance of informed consent.

“Informed consent” is a process by which individuals receive information about a genetic test that they may wish to select, are informed of both the potential benefits and risks associated with performing a genetic test, and provide legally binding consent for such a test to be performed on their provided sample. The term “consent” or “consenting” is also used to connote the process of obtaining legally binding informed consent by an individual. The term “assent” is used to connote the process by which an individual indicates their agreement but does not provide a legally binding informed consent. For example, minors generally are considered incapable of providing a legally binding consent, but may provide assent.

Sample consent forms used by leading genetics centers are shown in EXAMPLES 1 and 2. Standards of medical care and, in some states, state laws, require that an individual provide an informed consent for each genetic test that is performed. It is considered unethical, and in some states illegal, to perform a genetic test on an individuals samples without the explicit permission of that individual. In general, informed consent is provided for a single genetic test used designed to identify one or more than one specific variations within a gene. Thus, if consent is obtained to test for one or more than one variance within a gene and/or gene family in order to assess an individuals risk of a particular clinical outcome, it is not possible to test for variances in another gene should that gene be discovered to have a role in determining the individuals risk of that clinical outcome. It is apparent from EXAMPLES 1 and 2 that while residual DNA may be available in the laboratory, no provision is made for additional testing to provide a current assessment of genetic risk. In fact, retesting is not anticipated by such consents.

The invention comprises an integrated method for providing individuals with current assessments of genetic risk for a specific clinical outcome based on genetic tests and genomic research in which informed consent enables testing on a specific gene for specific variances, but also enables the testing to be performed in an integrated manner for additional variations in said gene or gene family that may be reported through genomic research as well as for variances in additional genes that may contribute to the genetic risk of a specific clinical outcome.

Current consent forms and current practice commonly prohibit retesting of patient samples. The term “retesting” refers to performing a genetic test for additional variances or variances in genes or performing a new analysis of genetic test results. The present invention provides forms and methods for performing retesting with genetic tests that are not explicitly listed in the original informed consent. Some consent forms allow samples to be used for research which may involve genetic tests only when the identity of the individual is separated from the sample and the sample and data is anonymized. It will be recognized by one of skill in the art that such samples are no longer useful for providing an individual with a current assessment of genetic risk since the process of anonymizing the sample is explicitly designed to make it impossible to ascribe specific findings of such research back to a specific individual, and any effort to even identify the research subject without their explicit consent would be considered unethical and potentially illegal.

The invention does not include retesting that is performed for purposes other than providing a current assessment of risk, specifically basic genomic research, epidemiological research, or the research and development of new biopharmaceutical products. It is significant that current policies and practices implemented by the genetics and ethics community specifically avert notification of individuals when such tests reveal variations that may be used to make an assessment of risk. Notification is not performed, and indeed is not considered ethical in such situations, because the existing methods for informed consent, testing, and record keeping associated with such research does not meet basic standards of medical care. For example, such testing is commonly performed in laboratories that do not meet CLIA standards, records are commonly maintained which do not adhere to the standards of medical records, for example those mandated by HIPA, there is no system or standards for monitoring, and there is no system or standards for notification. Moreover, informed consent and testing and record keeping is not integrated with any system for notification, retesting, or recounseling.

DNA banking is a process known in the art and is commonly performed as part of the testing process, particularly when there is an anticipation of the need to perform additional tests on the DNA sample. DNA banking is also performed for limited periods of time by many clinical and genetic laboratories for the purpose of quality control, and tests are often repeated if the results are equivocal or if control values fluctuate from established norms. DNA banking is also performed if additional testing may be performed on the individual's sample or to assemble collections of samples for research.

Informed consent is commonly required for DNA banking, and such consent commonly prohibits additional testing (other than for quality control purposes) on the sample without further informed consent. The invention provides integrated methods and systems that systematically monitor the progress of genomic research, notify individuals concerning newly discovered genes and variations that contribute to genetic risk, and enable retesting or recounseling to provide an assessment of risk. For the purpose of this invention, retesting refers to additional genetic tests performed on a sample (other than for quality control purposes) as well as additional analysis of genetic tests results. Retesting can commonly be performed on a sample that has undergone DNA banking or other samples from the patient that are stored using methods known in the art.

“Recounseling” is the process of providing an individual with information and guidance concerning their genetic risk of a clinical outcome based on new data derived through retesting. Ongoing genomic research is constantly revealing new variances, new genes, and new data for analysis that may alter an assessment of risk. The invention provides a system for monitoring genomic research, notifying individuals when such research related new information is reported, and providing a current assessment of risk through retesting and recounseling. The end result of this system is the opportunity to provide an individual with recounseling, allowing that individual to make healthcare and lifestyle decisions based on a current assessment of risk.

An embodiment of the method and systems of the present invention is the integration of systems for monitoring genomic research for reports and/or reference citations of genes and variations that contribute to a particular clinical outcome. The term “monitor” or “monitoring” refers to a system for scanning, reviewing and/or retrieving information from the medical literature relevant to providing a current assessment of genetic risk including, new variances, new genes, new analytical methods for genetic tests, or new clinical data related to an assessment of genetic risk based on genetic test results. Monitoring may be performed by individuals skilled in the art who are trained in a specific medical specialty using standard operating procedures to determine whether reports of new genes, variations, or analytical methods can change the assessment of risk sufficient to warrant notification of the individual.

An initial search of the literature, reports and/or citations is performed automatically on regular basis, for example daily, weekly, or monthly. The results of the automated search can be subject to review regarding the significance of the report in relation to an assessment of genetic risk using standard operating procedures and criteria. These standard operating procedures can assess the statistical significance of the report, the quantitative effect of the proposed genetic test on risk, the reproducibility of the reported results, the clinical significance of the proposed genetic test, its potential impact on individual healthcare and lifestyle decisions, and the economic implications of the proposed test. If standard criteria are satisfied, an assessment will be made regarding availability of the test, whether there are accepted algorithms for applying test results in an assessment of risk, whether validated testing methods have been established, whether the test is available from a CLIA-certified testing facility, and whether such facilities have sufficient capacity. If the test is available, prerequisites will be established for notifying individuals based on the characteristics of the individual for whom the test is indicated. Prerequisites can include the clinical diagnosis, its severity and pathology, family history, and previous genetic test results. Notification will be guided by systems that incorporate automated analysis and standard operating procedures.

Monitoring can also be performed using automated systems to review reports incorporating search terms for specific diseases, disease terms, numerical designation of diseases, specific symptoms, pathologies, or clinical outcomes. Monitoring may involve identifying reports on the basis of specific genes, gene families, pathways, or pathologies. Search functions, such as search engines and/or web browsers, capable of systematically publication lists, such as the published medical literature citations are known in the art. Monitoring may be triggered by the publication or release of journals that are known to have reports of genomic research.

Monitoring can be performed for clinical outcomes represented in individual records including the genetic risk of diseases as well as genetic factors that impact the response to therapy. For example, individuals who are receiving specific drugs as therapy for their disorders may be notified of citations, references, reports and/or genes that predict drug response or toxicity. As individuals are enrolled in the system and as information is added to the record, relevant reports from genomic research will be automatically identified and individuals will be notified as appropriate.

The term “notify” or “notifying” is known in the art and in reference to the present invention to communication with an individual concerning newly discovered genes and variations that contribute to genetic risk and the opportunity for a current assessment of risk based on retesting and recounseling. An individual may be notified by any communication medium known in the art for retrieving or reviewing information from a site on the Internet, email, fax, mail, telephone, or oral communication. In a specific embodiment, an individual is notified only when they access the system through an internet site or through alternative medium such as telephone, fax, or mail.

The Internet is recognized to be a potentially powerful medium for the delivery of healthcare. The term “e-health” refers to sites on the Internet that provide medical information, products, or services to individuals or to health care providers. More than 30% of all adults, and more than 70% of Internet users, visited e-health sites on the Internet in 1999. The Internet is heavily used by individuals in the 15-45 age bracket. This group that is said to be the least likely to contact health care professionals. As a result, many e-health sites have been established for providing general medical information and the sale of drugs, materials, equipment, or other products commonly available through healthcare providers or pharmacies. WebMD.com is an example of such a site.

As used herein, the terms “Internet” or “world wide web” are known in the art and refer to electronic networks, or elements of electronic networks, for the exchange of information between individuals and can include, for example, public systems such as the world wide web and public and/or private systems providing access to sites on said network including, for example, companies with private networks accessed by telephone, cable, wireless devises, or satellite and/or sites-providing portals for entry into any public or private network. The term “site” as used herein refers to software and hardware accessible through a URL (Universal Record Locator) or address on the Internet or world wide web and includes, for example, the concept, design, construction, appearance, organization, function, and content of materials posted and accessed at a particular URL. The term “secure site” as used herein refers to a site with software and/or hardware protective protocols and/or devices for privacy and security as are known in the art. It will be recognized by one of skill in the art that, in the interests of security, a site may be comprised of more than one linked address on the Internet and more than one server.

Methods for constructing and operating the site of the present invention including the software to create and operate the site and the hardware and Internet connections that make Internet sites are available over the Internet and are generally known in the art, are described in many books for lay and professional users of the Internet, and are available from commercial vendors. For example, FrontPage (Microsoft Corporation) is a simple computer program that can be used to create a web site. More sophisticated sites are generally created using computer languages such as HTML (and versions thereof) and Java by companies dedicated to webdesign and construction.

Web sites are commonly linked to various databases. For the present invention, the site can be linked to databases holding information regarding genetic testing, a database of individuals who access and use the invention web site, and databases containing personal genetic or medical records. Databases can be constructed and maintained using commercially available software such as Oracle. Patient data can also be tracked with commercial Customer Relations Management software such as for example software created by Eloyalty, Inc. Digital signatures can be used are obtained by using VeriSign Secure Digital ID. Payment(s) can be made by credit card or by way of Cyber\Cash. A payment system can also include software for correctly calculating sales tax and specifying shipping options, software such as, for example, Taxware and TanData. A site is commonly hosted on a server by an ISP (Internet Service Provider) such as, for example, UUNet, Genuity, ATT or Verio. The present invention's site can be hosted on a commercially available server such as a Compaq Enterprise Hosting NT system and run Microsoft Site Server Edition 3.0 and SQL Server Database. Various security systems and systems for encrypting data are known in the art and are generally available in major browser products. These systems are used to protect and secure of individual medical and financial records that may be available through the Internet providing privacy to the owners of the information. The present invention will benefit from advances in Internet software, hardware, and practices, the elements required to construct and operate the site anticipated by this and one of ordinary skill in the art would recognize that such factors are not limiting to the invention disclosed herein. It is recognized that the Internet has been used as a medium for dissemination of information regarding genomics including, for example, educational sites (such as accessexcellence.com), a compendium of inherited disorders (such as ncbi.omim.gov), and information about genetic tests and services (such as genetests.org and geneclinics.org). Many genetic testing services, universities, and companies maintain sites on the World Wide Web which provide description of the entities and the provided services., The presence of multiple sites of general and archival information regarding medicine, health, genetics, and genomic research that are available on the Internet are not limiting to the present invention. The present invention provides an integrated method for providing individuals with current assessments of genetic risk for a specific clinical outcome based on genetic tests and genomic research thereby providing an individual with current information and a current assessment of their genetic risk based on genomic research. The method integrates the complex, diverse and disclosure information on the Internet, reduces the technical nature of much of the genetic information and providing methods for quality control.

In another embodiment, the invention comprises a method for providing individuals with current assessments of genetic risk for a specific clinical outcome based on genetic tests and genomic research. A specific embodiment of this invention is an integrated method for performing a current assessment of genetic risk for an individual concerned about a specific clinical outcome integrating the following steps:

obtain consent of the patient for genetic testing and assessment of genetic risk for said outcome;

test for genes and variations known to be involved in genetic risk for said outcome;

counsel patient on test results and assessment of genetic risk;

record individual's identity, consent, contact information, clinical concerns, and genetic test results in a secure and private matter;

monitor genomic research for genes and variations that contribute to said clinical outcome;

notify individual concerning newly discovered genes and variations that contribute to genetic risk

retest for newly discovered genes and variations that contribute to genetic risk; and

recounsel patient on test results and current assessment of genetic risk.

In another embodiment, the invention provides a system for providing an individual with a current assessment of their genetic risk by integrating the steps of consent, testing, recording, monitoring, notifying, and retesting. An embodiment of this invention is a site on the Internet that provides an individual with a current assessment of genetic risk by integrating said steps.

In another embodiment, the invention comprises a system that provides a healthcare provider with a current assessment of genetic risk of an individual by integrating the steps of consent, testing, recording, monitoring, notifying, and retesting. Another embodiment of the invention comprises a site on the Internet that provides a healthcare provider with a current assessment of the genetic risk of an individual by integrating said steps. In yet another embodiment, the present invention comprises a computer system for providing a healthcare provider with a current assessment of the genetic risk of an individual by integrating said steps.

In another embodiment of the invention, a method is provided for performing a current assessment of genetic risk for an individual concerned about a specific clinical outcome. The method incorporates at least two of the following steps in an integrated manner:

obtain consent of the patient for genetic testing and assessment of genetic risk for said outcome;

test for genes and variations known to be involved in genetic risk for said outcome;

counsel the patient on test results and assessment of genetic risk;

record the individual's identity, consent, contact information, clinical concerns, and genetic test results in a secure and private matter;

monitor genomic research for genes and variations that contribute to said clinical outcome;

notify individual concerning newly discovered genes and variations that contribute to genetic risk

retest for newly discovered genes and variations that contribute to genetic risk; and

recounsel patient on test results and current assessment of genetic risk.

In another embodiment, the invention provides a method of performing a current assessment of genetic risk for an individual concerned about a specific clinical outcome incorporating three or more than three of the steps described herein. Additional embodiments of the invention integrate three, four or five of said steps.

The invention provides a system for providing an individual with a current assessment of their genetic risk through integrating two, or more than two, of the steps of consent, testing, recording, monitoring, notifying, and retesting. Another embodiment of the invention comprises a site on the Internet that provides an individual with a current assessment of genetic risk by integrating two, or more than two, of said steps.

In another embodiment, the invention comprises a system for providing a healthcare provider with a current assessment of the genetic risk of an individual by integrating two, or more than two, of the steps of consent, testing, recording, monitoring, notifying, retesting, and recounseling. In another embodiment of the invention comprises a site on the Internet that provides a healthcare provider with a current assessment of the genetic risk of an individual by integrating two, or more than two, of the steps provided herein above. In another embodiment of the present invention has a system for providing an individual with a current assessment of their genetic risk by integrating three, four or five of the steps provided herein above.

The invention provides an informed consent for performing a current assessment of genetic risk for an individual for a specific clinical outcome integrating the following steps:

test for genes and variations known to be involved in genetic risk for said outcome;

counsel the patient on test results and assessment of genetic risk;

record the individual's identity, consent, contact information, clinical concerns, and genetic test results in a secure and private matter;

monitor genomic research for genes and variations that contribute to said clinical outcome;

notify the individual concerning newly discovered genes and variations that contribute to genetic risk

retest the patient for newly discovered genes and variations that contribute to genetic risk; and

recounsel the patient on test results and current assessment of genetic risk.

In another embodiment, the invention provides an informed consent for performing a current assessment of genetic risk for an individual concerned about a specific clinical outcome incorporating at least two of the steps of counseling, testing, recording, monitoring, notifying, retesting, and recounseling. In yet another embodiment, the invention comprises an informed consent for performing a current assessment of genetic risk that integrates three, four, or five of the steps described herein above.

In another aspect of the invention, an informed consent is provided that allows the use of a system to provide an individual with a current assessment of their genetic risk that integrates the steps of counseling, testing, recording, monitoring, notifying, retesting, and recounseling. In another embodiment, the invention provides an informed consent that allows the use of a system that integrates two, three, four, or five of the steps as provided herein above, in providing an individual with a current assessment of genetic risk.

The invention provides an informed consent for using a web site to provide an individual with a current assessment of their genetic risk that integrates the steps of counseling, testing, recording, monitoring, notifying, retesting, and recounseling. Another embodiment of the invention is an informed consent that allows the use of a web site that integrates two, three, four, or five of the steps mentioned herein above in providing an individual with a current assessment of genetic risk.

In another embodiment of the invention has an informed consent which allows retesting for variances in a specific gene reported from genomic research. In another embodiment the invention comprises an informed consent which enables retesting for variances in a specified set of genes. In yet another embodiment, the invention comprises an informed consent which enables retesting for variances in a set of genes comprising a gene family, a gene pathway. Genes involved in a pathological process, and or a gene or genes that contribute to the risk of a specific clinical outcome.

In another embodiment of the invention, an informed consent is provided which enables retesting in conjunction with notification of the individual prior to said retesting. In another embodiment the invention comprises an informed consent which enables retesting with notification of the individual, where said retesting is integrated with recounseling, with notification and assent of said individual, and/or notification and assent of said individual where said retesting is integrated with recounseling. In another embodiment of the invention, notification is provided by accessing a secure web site, by email, telephone, fax, and/r other media. In another embodiment of the invention, notification and assent are provided by accessing a secure web site, by email, telephone, fax, or other media.

In another embodiment, the invention provides an informed consent for retesting an individuals sample without notification of the individual providing said consent, where said retesting is integrated with recounseling.

In another embodiment of the invention an informed consent is provided that allows notification of an individual when invention system monitoring of genomic research identifies new genetic tests that can be used to refine the assessment of the individuals genetic risk through retesting, retesting to be performed with assent, and/or retesting to be performed with further consent.

In another embodiment the invention comprises an informed consent for allowing a record to be maintained having and individual's identity, consent, contact information, indicated concerns about specific clinical outcomes, and genetic test results. The informed consent allows a record to be maintained with two, or more than two, of (individual's identity, consent, contact information, indicated concerns about specific clinical outcomes, and genetic test results). In another embodiment the invention comprises an informed consent that allows a record to be maintained with (individual's identity, consent, contact information, indicated concerns about specific clinical outcomes, and genetic test results) and a current assessment of genetic risk. In another embodiment, the informed consent allows record(s) to be maintained and accessed by the individual and/or health care professionals designated by the individual. In another embodiment one, or more than one, of the and individual's identity, consent, contact information, indicated concerns about specific clinical outcomes, and genetic test results of said record are maintained in a separate environment or medium that provides protection privacy and security of the information.

In another embodiment, the invention provides an informed consent for allowing a record to be posted on a secure site, on a secure site that may be accessed by the individual, and/or on a site that may be accessed by a health care professional designated by the individual.

In another embodiment, the informed consent allows the record to be posted on a site that can be accessed by the individual, where the site provides notification to the individual or to the appointed healthcare provider when genomic research identifies additional or new genetic tests that can be used to refine the assessment of genetic risk, and enables the individual to provide assent or consent for retesting.

In another embodiment, the invention provides a record having an individual's identity, consent, contact information, indicated concerns about specific clinical outcomes, and genetic test results, and/or identity, consent, contact information, indicated concerns about specific clinical outcomes, and genetic test results. The record can be accessed by an individual, and/or a health care professional designated by the individual. With regard to the record, one, or more than one, of identity, consent, contact information, indicated concerns about specific clinical outcomes, and genetic test results are maintained in a separate environment or medium that provides protection privacy and/or security.

In another embodiment, the invention comprises a record posted on a secure site. The secure site can be accessed by an individual, and/or by a health care professional designated by the individual.

In another embodiment, the invention comprises a record integrated with a system to monitor genomic research for genes and variations that contribute to the clinical outcome (in said record), and a system for notification of the individual and/or the healthcare provider when monitoring identifies new genetic tests that may be used for a current assessment of genetic risk. In a specific embodiment, the record is discrete and is accessed by the system independently of other records. In an alternative embodiment, the system maintains an index of records that pertain to a specific clinical outcome.

In another aspect the invention provides a record posted on a site integrated with a system that monitors posted genomic research results for genes and variations that contribute to a clinical outcome in said record. In another embodiment, the invention provides a record posted on a site integrated with a system to monitor genomic research for genes and variations that contribute to the clinical outcome in the record and comprises a system for notifying the individual and/or the health care provider when the invention system monitor identifies genetic tests that can be used for a current assessment of genetic risk.

In another embodiment the invention comprises a method for providing a current assessment of risk utilizing a record integrated with a system for monitoring genomic research for genes and variations that contribute to the clinical outcome of said record. The invention comprises a method for providing a current assessment of risk utilizing a record integrated with a system to monitor genomic research for genes and variations that contribute to the clinical outcome in said record and a notification system for contacting and/or notifying the individual and/or healthcare advisor when the system monitor identifies genetic tests that may be used for a current assessment of genetic risk.

In another embodiment, the invention comprises a system for providing an individual with a current assessment of their genetic risk utilizing a record integrated with a system to monitor genomic research for genes and variations that contribute to the clinical outcome in said record. In another embodiment, the invention provides an individual with a current assessment of their genetic risk utilizing a record integrated with a system that monitors genomic research for genes and variations that contribute to the clinical outcome in said record and also notifies the individual, and/or healthcare advisor of said record when the monitor identifies new genetic tests that may be used for a current assessment of genetic risk. In another embodiment, the invention comprises a system that provides a healthcare provider with a current assessment of the genetic risk of an individual utilizing a record integrated with a system that monitors genomic research for genes and variations that contribute to a clinical outcome in said record and also comprises a system for notifying the healthcare provider in said record when the monitor identifies new genetic test that can be used for a current assessment of genetic risk.

In another embodiment, the invention comprises a site for providing an individual with a current assessment of their genetic risk utilizing a record integrated with a system for monitoring genomic research for genes and variations that contribute to a clinical outcome in said record. The site provides an individual with a current assessment of their genetic risk utilizing a record integrated with a system for monitoring genomic research for genes and variations that contribute to the clinical outcome in said record and comprising a system for notifying the individual, and/or a healthcare provider in said record when the monitor identifies new genetic tests that may be used for a current assessment of genetic risk. In another embodiment, the invention comprises a site for providing a healthcare provider with a current assessment of the genetic risk of an individual utilizing a record integrated with a system that monitors genomic research for genes and variations that contribute to the clinical outcome in said record and comprises a system for notifying the healthcare provider in said record when the monitor identifies new genetic test that may be used for a current assessment of genetic risk.

In an additional embodiment, the invention comprises a method for monitoring genomic research for genes and variations that contribute to a clinical outcome. The monitoring can be performed for genes and variations that contribute to a clinical outcome in a record. Monitoring can be performed daily, weekly, every two weeks, three weeks and/or monthly, for reports with information concerning genes and variations that contribute to a clinical outcome in a record which are available to those skilled in the art. Monitoring is performed when there is a report, present, and/or available to those skilled in the art. Monitoring can also be performed on a continuous basis using systems that process new reports as they become available to those skilled in the art for information relevant to a clinical outcome in a record. In specific embodiments monitoring is performed for genes in a family, a pathway, and/or a pathological process.

In an additional embodiment, the invention comprises a site for monitoring genomic research for genes and variations that contribute to a clinical outcome. The clinical outcome can be listed in or part of a record. The site can be accessed by an individual and/or healthcare provider for the purpose of monitoring genomic research for genes and variations that contribute to a clinical outcome.

In an additional embodiment, the invention comprises a site for monitoring genomic research for genes and variations that contribute to a clinical outcome in a record integrated with a method for notification of the individual in said record when new genes or variations are identified.

In an additional embodiment, the invention comprises a system for monitoring genomic research for genes and variations that contribute to a clinical outcome. The clinical outcome can be part of a record. The monitoring of the invention can be performed daily, weekly, every two weeks, three weeks and/or monthly for genes and variations that contribute to a clinical outcome in a record. Monitoring is performed when there is a report, present, and/or available to those skilled in the art. Monitoring can also be performed on a continuous basis using systems that process new reports as they become available to those skilled in the art for information relevant to a clinical outcome in a record. In another embodiment, monitoring is performed for genes in a family, a pathway, and/or a pathological process.

In another embodiment, the invention comprises a system for notifying is an individual when monitoring of genomic research identifies new genetic tests that can be used to refine the assessment of the individual's genetic risk through retesting. Notification can be performed by telephone, email, fax, mail, and/or other medium. Notification can be provided, or accomplished by accessing a secure site. In a specific embodiment an individual is notified that there is a current assessment of risk, or new information which can be used to make a current assessment of risk, when that individual logs onto a specific web site, preferably logging on to the site using identifying information and a password that identifies the individual. In a preferred embodiment, the current assessment of risk is performed when an individual logs onto a specific web site if such assessment involves a change in the quantitative assessment of risk based on available genetic information. The system for providing a current assessment of risk for an individual concerned about a specific clinical outcome comprises a record including a patient's identity, consent record, contact information, clinical concerns and genetic test results, a subsystem for monitoring genomic research for genes and variations that contribute to said clinical outcome; and a subsystem for notifying said patient when the monitoring subsystem identifies new genetic test that may be used for updating said genetic test results. The system can comprise a secure web site.

In another embodiment, the system comprises a secure site that may be accessed by an individual, and/or healthcare advisor for notification that monitoring of genomic research identified new genetic tests that can be used to refine the assessment of the individuals genetic risk through retesting.

In another embodiment, the system includes a subsystem for notifying an individual when monitoring genomic research for genes and variations that contribute to a clinical outcome identifies new tests that can be used to refine the assessment of the individuals genetic risk, and can require retesting. Notification can be performed by telephone, email, fax, mail, and/or other medium.

In another embodiment, the system includes a site containing a record that can be accessed by the individual and/or the healthcare provider, a site can also provide notification when the monitor services of genomic research identify new genetic tests that can be used to refine the assessment of genetic risk, and provides a method for allowing the individual to provide assent for retesting. The site can be linked to systems that allow notification of an individual by email, telephone, fax, and/or other media.

In another embodiment the invention provides retesting integrated with the steps of recording, monitoring, and/or notifying. In such an embodiment the results of retesting are incorporated in said record. The results of retesting are provided to an individual, and/or provided to a healthcare provider designated by the individual. Retesting can be integrated with recounseling. Within an aspect of the invention recounseling can be integrated with the steps or recording, monitoring, notifying, and/or retesting. Recounseling can also be integrated with recording, where the results of recounseling are incorporated in a record.

The system can also comprise a contract for providing a current assessment of genetic risk to individual concerned about a specific clinical outcome incorporating two of the following steps in an integrated manner:

obtain consent of the patient for genetic testing and assessment of genetic risk for said outcome;

test for genes arid variations known to be involved in genetic risk for said outcome;

counsel patient on test results and assessment of genetic risk;

record individual's identity, consent, contact information, clinical concerns, and

genetic test results in a secure and private matter;

monitor genomic research for genes and variations that contribute to said clinical outcome;

notify individual concerning newly discovered genes and variations that contribute to genetic risk

retest for newly discovered genes and variations that contribute to genetic risk; and

recounsel patient on test results and current assessment of genetic risk.

In another embodiment, the invention provides contract for providing a current assessment of genetic risk for an individual concerned about a specific clinical outcome. The assessment can involve three or more than three, four, five or six of the steps as discussed herein above.

The contract of the invention involves at least one payment, or a predetermined price and incremental payments made on a regular basis. It is an aspect of the invention that payments can be made through a site.

In another embodiment, the invention comprises a secure site that can be accessed by an individual, where the site which can provide notification when genomic research identifies genetic tests that can be used to refine the assessment of genetic risk. The secure site can be accessed by an individual and allows the individual to provide assent or consent for retesting. The site can also be a secure site that enables an individual to provide payment for retesting.

### EXAMPLE 1

Informed Consent Currently Used for DNA Testing at the University of Pennsylvania

This state-of-the-art informed consent from a leading human genetics center demonstrates that although the possibility of retesting is acknowledged in informed consents, such testing is not anticipated in the current consent. Rather, the state-of-the-art informed consent cautions that samples may not be available for testing even if new, improved tests become available:

### EXAMPLE 3

Monitoring for Genes and Variances Involved in Arthritis

One element of monitoring is an automated search of MEDLINE for novel genetic associations with a disease, disorder or clinical outcome. A sample search is shown below using the online version of MEDLINE, PUBMED, and the Boolean search term “Arthritis and Genetics and Associations”. 1093 reports in MEDLINE as of Mar. 10, 2001 were identified and the 50 most recent reports are shown. Additional searching using methods known in the art to compensate for variations in natural language utilization will yield additional reports.

This example demonstrates the large number of reports on novel genetic associations being published in the medical literature. Many of these reports describe novel variances in genes known to be risk factors for arthritis, novel associations of genes with arthritis, or revised estimates of the impact of a gene or variance on disease risk. Also apparent from this example, is the complexity of monitoring the literature for this single disorder, both because of the large number of reports and the technical nature of such reports. In current practice, there are no systems for non-specialist providers or individuals to effectively monitor genomic research. The invention integrates a system for monitoring genomic research into methods and systems for providing individuals with a current assessment of genetic risk.

In practice, a search such as the one shown in this example would be performed automatically on regular basis, for example daily, weekly, or monthly. This automated search represents the first step in monitoring. The results of the automated search will be subject to expert review of the significance of the report on an assessment of genetic risk using standard operating procedures and criteria. These standard operating procedures will assess the statistical significance of the report, the quantitative effect of the proposed genetic test on risk, the reproducibility of the reported results, the clinical significance of the proposed genetic test, its potential impact on individual healthcare and lifestyle decisions, and the economic implications of the proposed test. If standard criteria are satisfied, an assessment will be made of the availability of the test, whether there are accepted algorithms for applying test results in an assessment of risk, whether validated testing methods have been established, whether the test is available from a CLIA-certified testing facility, and whether such facilities have sufficient capacity. If the test is available, prerequisites will be established for notifying individuals based on the characteristics of the individual for which the test is indicated. Prerequisites may include the clinical diagnosis, its severity and pathology, family history, and previous genetic test results.

PUBMED Search Results:

Search Terms: Arthritis and Genetics and Association

### EXAMPLE 5

Monitoring for Genes and Variances Involved in Cancer

Monitoring for genes involved in determining the genetic risk of cancer is particularly complex due to the diversity of different cancers and the involvement of extensive somatic mutation and germlne genetic variations in oncogenesis. Genetic aberrations are central to the process by which a normal cell becomes malignant. Somatic mutation is variation in the sequence of genes within a malignant cell reflecting mutations that have taken place in normal tissue during the course of oncogenesis. Germline variations are inherited variations in gene sequence, structure, function, and expression that are found throughout the body. While Oncologists are among the best-trained health care providers in the use of genetics and use this information in treating malignant disease. Primary care providers, who are in the position to help assess an individual's risk and implement healthcare or lifestyle changes to prevent cancer, are generally unable to monitor the extensive literature on the genetics of cancer and provide a current assessment of individual risk. Since there is clear evidence of the heritability of many cancers, the system described in the present invention could have a significant utility in preventing cancer.

PUBMED Search Results:

Search Terms: Cancer and Gene and Risk

DATE: Mar. 10, 2001

Items 1-100 of 5993

1 Bertario L, Russo A, Sala P, Eboli M, Giarola M, D'amico F, Gismondi V, Varesco L, Pierotti M A, Radice P. Genotype and phenotype factors as determinants of desmoid tumors in patients with familial adenomatous polyposis. Int J Cancer. Mar. 20, 2001;95(2): 102-107.

2 Tan W, Chen G F, Xing D Y, Song C Y, Kadlubar F F, Lin D X. Frequency of CYP2A6 gene deletion and its relation to risk of lung and esophageal cancer in the Chinese population. Int J Cancer. Mar. 20, 2001;95(2):96-101.

3 Mestiri S, Bouaouina N, Ahmed S B, Khedhaier A, Jrad B B, Remadi S, Chouchane L. Genetic variation in the tumor necrosis factor-alpha promoter region and in the stress protein hsp70-2.Cancer. Feb. 15, 2001;91(4):672-678.

4. Nishikawa A, Fujimoto T, Akutagawa N, Iwasaki M, Takeuchi M, Fujinaga K, Kudo R. p53 Polymorphism (codon-72) has no correlation with the development and the clinical features of cervical cancer. Int J Gynecol Cancer. September2000;10(5):402-407.

5 Kim C J, Um S J, Kim T Y, Kim E J, Park T C, Kim S J, Namkoong S E, Park J S. Regulation of cell growth and HPV genes by exogenous estrogen in cervical cancer cells. Int J Gynecol Cancer. March 2000;10(2):157-164.

6 Lee H, Greeley G H, Englander E W. Age-associated changes in gene expression patterns in the duodenum and colon of rats. Mech Ageing Dev. Apr. 15, 2001;122(4):355-371.

7 Borek C. Antioxidant Health Effects of Aged Garlic Extract. J Nutr. March 2001;131(3):1010s-1015S.

8 Fackenthal J D, Marsh D J, Richardson A L, Cummings S A, Eng C, Robinson B G, Olopade O I. Male breast cancer in Cowden syndrome patients with germline PTEN mutations. J Med Genet. March 2001;38(3):159-164.

9 Israel D A, Salama N, Arnold C N, Moss S F, Ando T, Wirth H P, Tham K T, Camorlinga M, Blaser M J, Falkow S, Peek R M. Helicobacter pylori strain-specific differences in genetic content, identified by microarray, influence host inflammatory responses. J Clin Invest. Mar. 1, 2001;107(5):611-620.

10 Deal C, Ma J, Wilkin F, Paquette J, Rozen F, Ge B, Hudson T, Stampfer M, Pollak M. Novel Promoter Polymorphism in Insulin-Like Growth Factor-Binding Protein-3Correlation with Serum Levels and Interaction with Known Regulators. J Clin Endocrinol Metab. Mar. 1, 2001;86(3):1274-1280.

11 Yokoyama A, Muramatsu T, Omori T, Yokoyama T, Matsushita S, Higuchi S, Maruyama K, Ishii H. Alcohol and aldehyde dehydrogenase gene polymorphisms and oropharyngolaryngeal, esophageal and stomach cancers in Japanese alcoholics. Carcinogenesis. March 20001;22(3):433-439.

12 Tomescu D, Kavanagh G, Ha T, Campbell H, Melton D W. Nucleotide excision repair gene XPD polymorphisms and genetic predisposition-to melanoma. Carcinogenesis. March 20001;22(3):403-408.

13 Petrowsky H, Sturm I, Graubitz O, Kooby D A, Staib-Sebler E, Gog C, Kohne C H, Hillebrand T, Daniel P T, Fong Y, Lorenz M. Relevance of Ki-67 antigen expression and K-ras mutation in colorectal liver metastases. Eur J Surg Oncol. February 2001;27(1):80-87.,

14 Kim H, Scorilas A, Katsaros D, Yousef G M, Massobrio M, Fracchioli S, Piccinno R, Gordini G, Diamandis E P. Human kallikrein gene 5 (KLK5) expression is an indicator of poor prognosis in ovarian cancer.Br J Cancer. March 2001;84(5):643-650.

15 Nishino H, Tokuda H, Murakoshi M, Satomi Y, Masuda M, Onozuka M, Yamaguchi S, Takayasu J, Tsuruta J, Okuda M, Khachik F, Narisawa T, Takasuka N, Yano M. Cancer prevention by natural carotenoids. Biofactors. 2000;13(1-4):89-94.

16 Cummings S, Olopade O. Predisposition testing for inherited breast cancer. Oncology (Huntingt). August 1998;12(8):1227-41; discussion 1241-2.

17 Khanna C M. Investigations of thyroid diseases—an update on diagnostic methods. J Assoc Physicians India. November 1998;46(11):948-52.

18 Heitmiller R F. Epidemiology, diagnosis, and staging of esophageal cancer. Cancer Treat Res. 2001; 105:375-86.

19 Rosas S L, Koch W, da Costa Carvalho M G, Wu L, Califano J, Westra W, Jen J, Sidransky D. Promoter hypermethylation patterns of p16,06-methylguanine-DNA-methyltransferase, and death-associated protein kinase in tumors and saliva of head and neck cancer patients. Cancer Res. Feb. 1, 2001;61 (3):939-42.

20 Slattery M L, Samowitz W, Ballard L, Schaffer D, Leppert M, Potter J D. A molecular variant of the APC gene at codon 1822: its association with diet, lifestyle, and risk of colon cancer. Cancer Res. Feb. 1, 2001;61(3):1000-4.

21 Tang Y M, Green B L, Chen G F, Thompson P A, Lang N P, Shinde A, Lin D X, Tan W, Lyn-Cook B D, Hammons G J, Kadlubar F F. Human CYP1B1 Leu432Val gene polymorphism: ethnic distribution in African-Americans, Caucasians and Chinese; oestradiol hydroxylase activity; and distribution in prostate cancer cases and controls. Pharmacogenetics. December 2000;10(9):761-6.

22 Nair U, Bartsch H. Metabolic polymorphisms as susceptibility markers for lung and oral cavity cancer.IARC Sci Publ. 2001;154:271-90.

23 Wild C P, Turner P C. Exposure biomarkers in chemoprevention studies of liver cancer.[ARC Sci Publ. 2001;154:215-22.

24 Ross R K. The role of molecular genetics in chemoprevention studies of prostate cancer.IARC Sci Publ. 2001;154:207-13.

25 Burn J, Chapman P D, Bishop D T, Smalley S, Mickleburgh I, West S, Mathers J C. Susceptibility markers in colorectal cancer.IARC Sci Publ. 2001;154:131-47.

26 Zheng W, Xie D, Cerhan J R, Sellers T A, Wen W, Folsom A R. Sulfotransferase 1A1 polymorphism, endogenous estrogen exposure, well-done meat intake, and breast cancer risk. Cancer Epidemiol Biomarkers Prev. February 2001;10(2):89-94.

27 Stern M C, Umbach D M, van Gils C H, Lunn R M, Taylor J A. DNA repair gene XRCC1 polymorphisms, smoking, and bladder cancer risk. Cancer Epidemiol Biomarkers Prev. February 2001;10(2):125-31.

28 Ratnasinghe D, Yao S X, Tangrea J A, Qiao Y L, Andersen M R, Barrett M J, Giffen C A, Erozan Y, Tockman M S, Taylor P R. Polymorphisms of the DNA repair gene XRCC1 and lung cancer risk. Cancer Epidemiol Biomarkers Prev. February 2001;10(2):119-23.

29 Burmeister T, Thiel E. Molecular genetics in acute and chronic leukemias. J Cancer Res Clin Oncol. February 2001;127(2):80-90. Review.

30 Runnebaum I B, Stickeler E. Epidemiological and molecular aspects of ovarian cancer risk. J Cancer Res Clin Oncol. February 2001;127(2):73-9. Review.

31 Berstein L M, Imyanitov E N, Suspitsin E N, Grigoriev M Y, Sokolov E P, Togo A, Hanson K P, Poroshina T E, Vasiljev D A, Kovalevskij A Y, Gamajunova V B. CYP19 gene polymorphism in endometrial cancer patients. J Cancer Res Clin Oncol. February 2001; 127(2): 135-8.

32 Bennett L M, McAllister K A, Ward T, Malphurs J, Collins N K, Seely J C, Davis B J, Wiseman R W. Mammary tumor induction and premature ovarian failure in ApcMin mice are not enhanced by Brca2 deficiency. Toxicol Pathol. January-February 2001;29(1):117-25.

33 Shanahan F. Relation between colitis and colon cancer. Lancet. Jan. 27, 2001;357(9252):246-7. No abstract available.

34 Feigelson H S, McKean-Cowdin R, Coetzee G A, Stram D O, Kolonel L N, Henderson B E. Building a multigenic model of breast cancer susceptibility: CYP17 and HSD17B1 are two important candidates. Cancer Res. Jan. 15, 2001;61(2):785-9.

35 Campbell-Thompson M, Lynch I J, Bhardwaj B. Expression of estrogen receptor (ER) subtypes and ERbeta isoforms in colon cancer. Cancer Res. Jan. 15, 2001;61 (2):632-40.

36 Seewaldt V L, Mrozek K, Dietze E C, Parker M, Caldwell L E. Human papillomavirus type 16 E6 inactivation of p53 in normal human mammary epithelial cells promotes tamoxifen-mediated apoptosis. Cancer Res. Jan. 15, 2001;61(2):616-24.

37 Yang W C, Mathew J, Velcich A, Edelmann W, Kucherlapati R, Lipkin M, Yang K, Augenlicht L H. Targeted inactivation of the p21(WAF1/cip1) gene enhances Apc-initiated tumor formation and the tumor-promoting activity of a Western-style high-risk diet by altering cell maturation in the intestinal mucosal. Cancer Res. Jan. 15, 2001;61(2):565-9.

38 Takeshita T, Morimoto K, Yamaguchi N, Watanabe S, Todoroki I, Honjo S, Nakagawa K, Kono S. Relationships between cigarette smoking, alcohol drinking, the ALDH2 genotype and adenomatous types of colorectal polyps in male self-defense force officials. J Epidemiol. November 2000;10(6):366-71.

39 Rautelin H I, Oksanen A M, Karttunen R A, Seppala K M, Virtamo J R, Aromaa A J, Kosunen T U. Association of CagA-positive infection with Helicobacter pylori antibodies of IgA class. Ann Med. December 2000;32(9):652-6.

40 Loriot M A, Rebuissou S, Oscarson M, Cenee S, Miyamoto M, Ariyoshi N, Kamataki T, Hemon D, Beaune P, Stucker I. Genetic polymorphisms of cytochrome P450 2A6 in a case-control study on lung cancer in a French population. Pharmacogenetics. 2001 February; 11(1):39-44.

41 Woodson K, Mason J, Choi S W, Hartman T, Tangrea J, Virtamo J, Taylor PR, Albanes D. Hypomethylation of p53 in peripheral blood DNA is associated with the development of lung cancer. Cancer Epidemiol Biomarkers Prev. January 2001;10(1):69-74.

42 Willett W C. Diet and cancer: one view at the start of the millennium. Cancer Epidemiol: Biomarkers Prev. January 2001;10(1):3-8.

43 Stal O, Borg A, Ferno M, Kallstrom A C, Malmstrom P, Nordenskjold B. ErbB2 status and the benefit from two or five years of adjuvant tamoxifen in postmenopausal early stage breast cancer. Ann Oncol. December 2000;11(12):1545-50.

44 Russo A, Zanna I, Tubiolo C, Migliavacca M, Bazan V, Latteri M A, Tomasino R M, Gebbia N. Hereditary common cancers: molecular and clinical genetics. Anticancer Res. November-December 2000;20(6C):4841-51. Review.

45 Kodama M, Kodama T, Murakami M. Oncogene activation and tumor suppressor gene inactivation find their sites of expression in the changes in time and space of the age-adjusted cancer incidence rate. In Vivo. November-December 2000;14(6):725-34.

46 Olufunmilayo I, Olopade M D, Fackenthal J D. Breast cancer genetics. Implications of clinical practice]Hematol Oncol Clin North Am. June 2000;14(3):705-25. Review.

47 Belogubova E V, Togo A V, Kondrat'eva T V, Lemekhov V G, Barchuk A S, Romanenko S M, Khanson K P, Imianitov E N. [Polymorphism of the GSMT1 gene in lung cancer resistance and susceptibility].Vopr Onkol. 2000;46(5):549-54. Russian.

48 Eder E, Schuler D. An approach to cancer risk assessment for the food constituent 2-hexenal on the basis of 1,N2-propanodeoxyguanosine adducts of 2-hexenal in vivo. Arch Toxicol. December 2000;74(10):642-8.

49 Gattas G J, Soares-Vieira J A. Cytochrome P450-2E1 and glutathione S-transferase mu polymorphisms among Caucasians and mulattos from Brazil. Occup Med (Lond). September 2000;50(7):508-11.

50 Kim Y I, Baik H W, Fawaz K, Knox T, Lee Y M, Norton R, Libby E, Mason J B. Effects of folate supplementation on two provisional molecular markers of colon cancer: a prospective, randomized trial. Am J Gastroenterol. January 2001;96(1):184-95.

51 Xia H H, Talley N J. Apoptosis in gastric epithelium induced by Helicobacter pylori infection: implications in gastric carcinogenesis. Am J Gastroenterol. January 2001;96(1):16-26. Review.

52 Jackson P E, Qian G S, Friesen M D, Zhu Y R, Lu P, Wang J B, Wu Y, Kensler T W, Vogelstein B, Groopman J D. Specific p53 mutations detected in plasma and tumors of hepatocellular carcinoma patients by electrospray ionization mass spectrometry. Cancer Res. Jan. 1, 2001;61(1):33-5.

53 Zochbauer-Muller S, Fong K M, Virmani A K, Geradts J, Gazdar A F, Minna J D. Aberrant promoter methylation of multiple genes in non-small cell lung cancers. Cancer Res. Jan. 1, 2001;61(1):249-55.

54 Lubet R A, Zhang Z, Wiseman R W, You M. Use of p53 transgenic mice in the development of cancer models for multiple purposes. Exp Lung Res. December 2000;26(8):581-93. Review.

55 Snijders A M, Meijer G A, Brakenhoff R H, van den Brule A J, van Diest P J. Microarray techniques in pathology: tool or toy? Mol Pathol. December 2000;53(6):289-94. Review.

56 Machackova E, Foretova L, Navratilova M, Valik D, Claes K, Messiaen L. A high occurrence of BRCA1 and BRCA2 mutations among Czech hereditary breast and breast-ovarian cancer families. Cas Lek Cesk. Oct. 11, 2000;139(20):635-7.

57 Li J, Wang J, Guo Z. [Study of Bax gene in laryngeal carcinoma and it's clinical relationship].Lin Chuang Er Bi Yan Hou Ke Za Zhi. January 1998;12(1):16-8. Chinese.

58 Kinjo J.[Phytoestrogens].Nippon Rinsho. December 2000;58(12):2434-8. Review. Japanese.

59 Thune I, Smeland S. [No title available].Tidsskr Nor Laegeforen. Nov. 10, 2000;120(27):3296-301. Norwegian.

60 Sankaranarayanan K. Cancer predisposition, radiosensitivity and the risk of radiation-induced cancers: biological aspects and computational modeling. Radiat Res. December 2000;154(6):724-5;discussion 726-7.

61 McBride C M, Halabi S, Bepler G, Lyna P, Mcintyre L, Lipkus I, Albright J, O'Briant K. Maximizing the motivational impact of feedback of lung cancer susceptibility on smokers' desire to quit. J Health Commun. July-September 2000;5(3):229-41.

62 Olsen J H, Hahnemann J M, Borresen-Dale A L, Brondum-Nielsen K, Hammarstrom L, Kleinerman R, Kaariainen H, Lonnqvist T, Sankila R, Seersholm N, Tretli S, Yuen J, Boice J D, Tucker M. Cancer in Patients With Ataxia-Telangiectasia and in Their Relatives in the Nordic Countries. J Natl Cancer Inst. Jan. 17, 2001;93(2):121-127.

63 Duncan L M, Deeds J, Cronin F E, Donovan M, Sober A J, Kauffman M, McCarthy J J. Melastatin Expression and Prognosis in Cutaneous Malignant Melanoma. J Clin Oncol. Jan. 15, 2001;19(2):568-576.

64 Terdiman J P, Gum J R, Conrad P G, Miller G A, Weinberg V, Crawley S C, Levin T R, Reeves C, Schmitt A, Hepburn M, Sleisenger M H, Kim Y S. Efficient Detection of Hereditary Nonpolyposis Colorectal Cancer Gene Carriers by Screening for Tumor Microsatellite Instability Before Germline Genetic Testing.Gastroenterology. January 2001;120(1):21-30.

65 Figer A, Irmin L, Geva R, Flex D, Sulkes J, Sulkes A, Friedman E. The rate of the 6174delT founder Jewish mutation in BRCA2 in patients with non-colonic gastrointestinal tract tumours in Israel.Br J Cancer. February 2001;84(4):478-481.

66 Pacak K, Linehan W M, Eisenhofer G, Walther M M, Goldstein D S. Recent advances in genetics, diagnosis, localization, and treatment of pheochromocytoma. Ann Intern Med. Feb. 20, 2001;134(4):315-29.

67 Lim H N, Meyts E R, Skakkebaek N E, Hawkins J R, Hughes I A. Genetic analysis of the INSL3 gene in patients with maldescent of the testis. Eur J Endocrinol. February 2001; 144(2): 129-137.

68 Kadkhodayan S, Coin F, Salazar E P, George J W, Egly J, Thompson L H. Codominance associated with overexpression of certain XPD mutations. Mutat Res. Mar. 7, 2001;485(2):153-168.

69 Brekelmans C T, Seynaeve C, Bartels C C, Tilanus-Linthorst M M, Meijers-Heijboer E J, Crepin C M, van Geel A N, Menke M, Verhoog L C, van Den Ouweland A, Obdeijn I M, Klijn J G. Effectiveness of Breast Cancer Surveillance in BRCA½ Gene Mutation Carriers and Women With High Familial Risk. J Clin Oncol. Feb. 15, 2001;19(4):924-930.

70 Chen Z, Karaplis A C, Ackerman S L, Pogribny I P, Melnyk S, Lussier-Cacan S, Chen M F, Pai A, John S W, Smith R S, Bottiglieri T, Bagley P, Selhub J, Rudnicki M A, James S J, Rozen R. Mice deficient in methylenetetrahydrofolate reductase exhibit hyperhomocysteinemia and decreased methylation capacity, with neuropathology and aortic lipid deposition. Hum Mol Genet. Mar. 1, 2001;10(5):433-443.

71 Burnouf D Y, Miturski R, Nagao M, Nakagama H, Nothisen M, Wagner J, Fuchs R P. Early detection of 2-amino-1-methyl-6-phenylimidazo (4,5-b)pyridine(PhIP)-induced mutations within the Apc gene of rat colon. Carcinogenesis. February 2001;22(2):329-335.

72 Williams J A. Single nucleotide polymorphisms, metabolic activation and environmental carcinogenesis: why molecular epidemiologists should think about enzyme expression. Carcinogenesis. February 2001;22(2):209-214.

73 Julian-Reynier C, Sobol H, Sevilla C, Nogues C, Bourret P. Uptake of hereditary breast/ovarian cancer genetic testing in a French national sample of BRCA1 families. The French Cancer Genetic Network. Psychooncology. November-December 2001;9(6):504-10.

74 Hopwood P, Lee A, Shenton A, Baildam A, Brain A, Lalloo F, Evans G, Howell A. Clinical follow-up after bilateral risk reducing (‘prophylactic’) mastectomy: mental health and body image outcomes. Psychooncology. November-December 2000;9(6):462-72.

75 Moore D F, Chatterjee N, Pee D, Gail M H. Pseudo-likelihood estimates of the cumulative risk of an autosomal dominant disease from a kin-cohort study. Genet Epidemiol. February 20001;20(2):210-227.

76 Misra R R, Tangrea J A, Virtamo J, Ratnasinghe D, Andersen M R, Barrett M, Taylor P R, Albanes D. Variation in the promoter region of the myeloperoxidase gene is not directly related to lung cancer risk among male smokers in Finland. Cancer Lett. Mar. 26, 2001;164(2):161-167.

77 Zhu M, Chapman W G, Oberley M J, Wasserman W W, Fahl W E. Polymorphic electrophile response elements in the mouse glutathione S-transferase GSTa1 gene that confer increased induction. Cancer Lett. Mar. 26, 2001;164(2):113-118.

78 Mao H, Liu H, Fu X, Fang Z, Abrams J, Worsham M J. Loss of nm23 expression predicts distal metastases and poorer survival for breast cancer. Int J Oncol. March 2001;18(3):587-591.

79 Risch H A, McLaughlin J R, Cole D E, Rosen B, Bradley L, Kwan E, Jack E, Vesprini D J, Kuperstein G, Abrahamson J L, Fan I, Wong B, Narod S A. Prevalence and Penetrance of Germline BRCA1 and BRCA2 Mutations in a Population Series of 649 Women with Ovarian Cancer. Am J Hum Genet. March 2001;68(3):700-710.

80 Eeles R A. Future possibilities in the prevention of breast cancer: Intervention strategies in BRCA1 and BRCA2 mutation carriers. Breast Cancer Res. 2000;2(4):283-290.

81 Strong T V. Gene therapy for carcinoma of the breast: Genetic immunotherapy. Breast Cancer Res. 1999;2(1):15-21.

82 Lalloo F, Evans D G. The pathology of familial breast cancer: Clinical and genetic counseling implications of breast cancer pathology. Breast Cancer Res. 1999;1 (1):48-51.

83 Loktionov A, Scollen S, McKeown N, Bingham S A. Gene-nutrient interactionsdietary behaviour associated with high coronary heart disease risk particularly affects serum LDL cholesterol in apolipoprotein E epsilon4-carrying free-living individuals.Br J Nutr. December 2000;84(6):885-90.

84 Nakajima K, Sasaki M, Nojima D, Oh B R, Ishii N, Miura K, Dahiya R. TUMOR NECROSIS FACTOR-alpha GENE MUTATIONS AND GENOTYPE CHANGES IN RENAL CELL CARCINOMA.J Urol. February 2001;165(2):612-615.

85 Leggett B A, Devereaux B, Biden K, Searle J, Young J, Jass J. Hyperplastic polyposisassociation with colorectal cancer. Am J Surg Pathol. 2001 February;25(2):177-84.

86 Tavtigian S V, Simard J, Teng D H, Abtin V, Baumgard M, Beck A, Camp N J, Carillo A R, Chen Y, Dayananth P, Desrochers M, Dumont M, Farnham J M, Frank D, Frye C, Ghaffari S, Gupte J S, Hu R, Iliev D, Janecki T, Kort E N, Laity K E, Leavitt A, Leblanc G, McArthur-Morrison J, Pederson A, Penn B, Peterson K T, Reid J E, Richards S, Schroeder M, Smith R, Snyder S C, Swedlund B, Swensen J, Thomas A, Tranchant M, Woodland A M, Labrie F, Skolnick M H, Neuhausen S, Rommens J, Cannon-Albright L A., OMIMA candidate prostate cancer susceptibility gene at chromosome 17p.Nat Genet. February 2001;27(2):172-180.

87 Kim J W, Roh J W, Park N H, Song Y S, Kang S B, Lee H P. Polymorphism of TP53 codon 72 and the risk of cervical cancer among Korean women. Am J Obstet Gynecol. January 2001;184(2):55-58.

88 Schnakenberg E, Breuer R, Werdin R, Dreikorn K, Schloot W. Susceptibility genesGSTM1 and GSTM3 as genetic risk factors in bladder cancer. Cytogenet Cell Genet. 2000;91(1-4):234-238.

89 Cussenot O, Valeri A. Heterogeneity in genetic susceptibility to prostate cancer. Eur. J. Intern. Med. February 2001;12(1):11-16.

90 Owen R J, Peters T M, Varea R, Teare E L, Saverymuttu S. Molecular epidemiology of Helicobacter pylori in England: prevalence of cag pathogenicity island markers and IS605 presence in relation to patient age and severity of gastric disease. FEMS Immunol Med Microbiol. February 2001;30(1):65-71.

91 Thompson D, Easton D. Variation in cancer risks, by mutation position, in BRCA2 mutation carriers. Am J Hum Genet. February 2001;68(2):410-9.

92 Gronberg H, Ahman A K, Emanuelsson M, Bergh A, Damber J E, Borg A A. BRCA2 mutation in a family with hereditary prostate cancer. Genes Chromosomes Cancer. March 2001;30(3):299-301.

93 Grant D J, Bell D A. Bilirubin UDP-glucuronosyltransferase 1A1 gene polymorphisms Susceptibility to oxidative damage and cancer? Mol Carcinog. December 2000; 29(4): 198-204.

94 Ruttenber A J, Harrison L T, Baron A, McClure D, Glanz J, Quillin R, O'Neill J P, Sullivan L, Campbell J, Nicklas J A. hprt mutant frequencies, noripulmonary malignancies, and domestic radon exposure: “postmortem” analysis of an interesting hypothesis. Environ Mol Mutagen. 2001;37(1):7-16.

95 Maugard C M, Charrier J, Pitard A, Campion L, Akande O, Pleasants L, Ali-Osman F. Genetic polymorphism at the glutathione S-transferase (GST) P1 locus is a breast cancer risk modifier. Int J Cancer. Feb. 1, 2001;91(3):334-9.

96 Russo J, Hu Y F, Silva I D, Russo I H. Cancer risk related to mammary gland structure and development. Microsc Res Tech. Jan. 15, 2001;52(2):204-223.

97 de Haas V, Oosten, L, Dee R, Verhagen O J, Kroes W, van den Berg H, van der Schoot C E. Minimal residual disease studies are beneficial in the follow-up of TEL/AML1 patients with B-precursor acute lymphoblastic leukaemia.Br J Haematol. December 2000;111 (4):1080-6.

98 Parshad R, Sanford K K. Radiation-induced chromatid breaks and deficient DNA repair in cancer predisposition. Crit Rev Oncol Hematol. February 2001;37(2):87-96.

99 Brandt B H, Schmidt H, de Angelis G, Zanker K S. Predictive laboratory diagnostics in oncology utilizing blood-borne cancer cells—current best practice and unmet needs. Cancer Lett. January 2001;162 Suppl:S11-S16.

100 Nam R K, Toi A, Vesprini D, Ho M, Chu W, Harvie S, Sweet J, Trachtenberg J, Jewett M A, Narod SA. V89L polymorphism of type-2,5-alpha reductase enzyme gene predicts prostate cancer presence and progression. Urology. January 2001;57(1):199-204.

### EXAMPLE 6

Genetic Tests Predicting Common Diseases

Most common diseases are considered to be multifactorial or polygenic, meaning that many different genes may contribute to the risk of the disorder. Genetic testing is performed for genes known to contribute to these disorders so that the environmental factors which contribute to the disease can be avoided or treated through changes in lifestyle or healthcare. An assessment of risk can be made on the basis of these tests. Individuals who undergo genetic testing for these genes would benefit from the current invention which enables a current assessment of risk to be made based on new genes and variations reported from genomic research. Examples of genetic tests that can be used to assess the risk of common disorders that are currently available include without limitation: (this list derived in part from genetests.org).

| | |
|---|---|
| Disorder | Genetic test |
| Cancer | Breast Cancer (BRCA1)*; BRCA1; Ovarian Cancer |
| | (BRCA1) Breast Cancer (BRCA2)*; BRCA2; |
| | Ovarian Cancer (BRCA2) |
| | p53 |
| | p21 |
| | p16 |
| | Ataxia Telangectasia |
| | Familial Colorectal Cancer; Familial Colon Cancer |
| | Medullary Thyroid Carcinoma; MTC |
| Alzheimer's Disease | Apolipoprotein E |
| | β amyloid precursor protein |
| | protein t |
| | presenilin-1, presenilin-2 |
| | α 2-macroglobulin |
| | a 1-antichymotrypsin |
| Heart attack, stroke | Apolipoprotein E |
| | Lipoprotein lipase |
| | LDL receptor |
| | MTHFR |
| ALS | Superoxide Dismutase (SOD) |
| COPD | α 1-antitrypsin (AAT) |
| Anemia | hemoglobin S |
| | hemoglobin C |
| | thalassemia (α) |
| | thalassemia (β) |
| | G-6 PD |
| Liver failure | Hemochromatosis |
| Spina Bifida | MTHFR |
| Arthritis | HLA-B, HLA-D |
| Periodontal disease | IL-1 |

### EXAMPLE 7

Genetic Tests Predictive of Drug Response

Variations in genes that affect the metabolism of drugs can increase drug levels, drug toxicity and drug interactions. Genetic tests can be used to avoid drugs that have a higher probability of toxicity and individualize the dose to maximize the therapeutic benefit while minimizing toxicity. The following are examples, without limitation, of tests that can be used to guide the safety and appropriate application of important drugs. Individuals who undergo genetic testing for these genes would benefit from the current invention which enables a current assessment of risk to be made based on new genes and variations reported from genomic research. (This list derived in part from genetests.org).

| | |
|---|---|
| CYP1A1 | Chlorinated benzenes (environmental toxin) |
| CYP1A2 | Caffeine, phenacetin, warfarin, Erythromycin, |
| | Ropivacaine, Haloperidol, antipyrine, theophylline, |
| | Paracetamol |
| CYP2C8 | TCA, Diazepam, Hexabarbitone |
| CYP2C9/10 | Phenytoin, S-warfarin, Diclofenac, Tolbutamide |
| CYP2C19 | Mephenytoin, Diazepam (Valium), TCA |
| CYP2D6 | Debrisoquine, Codeine, Dextrometorphan, b- |
| | blockers, SSRIs, others |
| CYP2E1 | Paracetamol, Isoflurane, Sevoflurane, |
| | Methoxyflurane, Enflurane, Trichorethylene |
| CYP3A4 | Nifedipine, Dextrometorphan, Alfentanil, Sufentanil, |
| | Fentanyl, Erythromycin, Lignocaine, Ropivacaine, |
| | Midazolam, Codeine, Granisetron, Hydrocortisone |
| CYP3A5 | Caffeine, Diltiazem |
| CYP3A7 | Midazolam |
| CYP17 | Pregnolone |
| CYP19 | Testosterone |
| CYP21A2 | 17-hydroxyprogesterone |

Variations in genes that affect drug targets and drug response may affect the safety and efficacy of a drug. Genetic tests can be used to avoid drugs that have a higher probability of toxicity and individualize the dose to maximize the therapeutic benefit while minimizing toxicity. Individuals who undergo genetic testing for these genes would benefit from the current invention which enables a current assessment of risk to be made based on new genes and variations reported from genomic research.

| | |
|---|---|
| Factor V | Oral contraceptives |
| Prothrombin | Oral contraceptives |
| TPMT (thiopurine methyltransferase) | Azothioprine, mercaptopurine |
| | (purine analogues) |
| 5′ lipoxegenase | Zilutin (5′ lipoxegenase |
| | inhibitors) |
| CETP (cholesterol ester transfer protein) | Pravastatin, others (statins) |
| ApoE (apolipoprotein E) | Tacrine (cholinesterase |
| | inhibitors, muscarinic agonists) |
| G-6 PD (glucose 6 phosphase | sulfur drugs |
| dehydrogenase) | |
| pseudocholinesterase | pseudocholinesterase inhibitors |
| □-receptor | Isoproterenol (□-agonists) |
| Serotonin transporter | SSRI antidepressants (Prozac, |
| | Pindolol and others) |
| acetyltransferase | isoniazid, others |
| ADH(2h) (aldehyde dehydrogenase) | alcohol |
| ACE (angiotensin converting enzyme) | Enalpril, others |
| opioid receptors | Endorphins, morphine |

### EXAMPLE 8

Genetic Tests for Monogenic Disoreders Disease

A large number or inherited genetic diseases are caused by well-characterized mutations in genes that impair the function of a gene or cause a gene to have dominant, adverse effects. Many of these tests are performed in academic, hospital clinical laboratories or in the research laboratories of scientists who study these disorders. The following is partial list of genetic tests for inherited genetic diseases. Individuals who undergo genetic testing for these genes would benefit from the current invention that may enable identification of additional genes that affect the expression of the disorder in the individual. This list was derived, in part, from http://genetests.org.

Achondroplasia*

Adenosine Monophosphate Deaminase 1*; AMPD1; Exercise-induced Myopathy

Adrenoleukodystrophy, X-linked*; Addison Disease and Cerebral Sclerosis;

Adrenomyeloneuropathy; Adrenoleukodystrophy, Recessive*; Neonatal

Adrenoleukodystrophy

Alpha Thalassemia

Alpha-1-Antitrypsin Deficiency

Amyloidosis Type I*; Amyloid Polyneuropathy, Andrade or Portugese Type; Amyloidosis, Portugese Type

Amyloidosis, Swedish Type

Angelman Syndrome

Azoospermia*; Oligospermia (CFTR)

Bloom Syndrome*

Canavan Disease

Carnitine Palmitoyltransferase Deficiency*; CPT I Deficiency; CPT II Deficiency

Carnitine Deficiency, Systemic*

Charcot-Marie-Tooth Disease, X-linked*; CMTX; HMSN, X-linked; Hereditary Motor and Sensory Neuropathy, Charcot-Marie-Tooth Disease,

Citrullinemia*

Congenital Bilateral Absence of the Vas Deferens*; CBAVD

Congenital Adrenal Hyperplasia*; 21-Hydroxylase Deficiency; CAH

Cystic Fibrosis*; CF

Cytochrome C Oxidase Deficiency*; COX Deficiency

Dentatorubral-Pallidoluysian Atrophy*; DRPLA

Duchenne Muscular Dystrophy*; BMD, included; Becker Muscular Dystrophy, included;

DMD Dystonia Type I*; Torsion Dystonia 1, Dominant

Early Onset Familial Alzheimer Disease*; AD1; AD3; AD4; Alzheimer Disease, Type 1; Alzheimer Disease, Type

Factor V Leiden Mutation*; Resistance to Activated Protein C; Thrombophilia V(Protein C Resistance); Thrombosis Risk Factor (Factor V Leiden)

Fragile X Syndrome*; FRAXA; Martin-Bell syndrome

Friedreich Ataxia

Galactosemia*; Galactose-1-Phosphate Uridyltransferase Deficiency

Gaucher Disease*; Glucocerebrosidase Deficiency

Genotypic Gender Assignment*; XX/XY Gender Assignment

Glycogen Storage Disease Type III*; Cori Disease; Debrancher Deficiency; Forbe Disease

Glycogen Storage Disease Type VII*; PFK Deficiency; Phosphofructokinase Deficiency; Tarui Disease

Glycogen Storage Disease Type IV*; Brancher Deficiency

Glycogen Storage Disease Type V*; McArdle Syndrome

Glycogen Storage Disease Type II*; Pompe Disease

Hemochromatosis

Hemoglobin E*

Hemoglobin C*; SC Disease; Sickle Cell Disease (Hemoglobin C)

Hemoglobin S*; Sickle Cell Anemia; Sickle Cell Disease (Hemoglobin S)

Hemophilia A*; Factor VIII Deficiency

Hemophilia B*; Christmas Disease; Factor 1×Deficiency

Hereditary Motor and Sensory Neuropathy, Dominant (Type 1)

Hereditary Neuropathy with Liability to Pressure Palsies*; HNPP

Huntington Disease*; HD

Hydrocephalus, X-linked*; Aqueductal Stenosis,

Hypochondroplasia

Kennedy Disease*; SBMA; Spinal and Bulbar Muscular Atrophy

Lactate Dehydrogenase Deficiency*; LDH Deficiency

Late Onset Familial Alzheimer Disease*; AD2; AD5; Alzheimer Disease (Apolipoprotein E); Alzheimer Disease, Medium Chain Acyl-CoA Dehydrogenase Medullary Thyroid Carcinoma*; MTC

Leber Hereditary Optic Neuropathy

Marfan Syndrome*

Medium Chain Acyl-CoA Dehydrogenase Deficiency*; MCAD Deficiency

Mitochondrial Myopathy*; Kearns-Sayre Syndrome; LHON; Leigh Disease; MELAS;

MERRF; NARP

MTHFR Thermolabile Variant*; Cardiovascular Risk Factor, Neural Tube Defect Risk Factor, Preeclampsia Risk Factor, Thrombosis Risk Factor

Multiple Endocrine Neoplasia Type 2B/3*; MEN2B; MEN3

Multiple Endocrine Neoplasia Type 2A*; MEN2A

Myotonic Dystrophy*; Steinert Disease

Neurofibromatosis Type II*; NF2

Neurofibromatosis Type I*; NF1; Von Recklinghausen Disease

Niemann-Pick Disease*

Norrie Disease*

Parentage Testing*; Maternity Testing; Paternity Testing

Phenylketonuria, Phenylalanine, Hydroxylase Deficiency

Phosphoglycerate Mutase Deficiency*; PGAM Deficiency

Phosphoglycerate Kinase Deficiency*; PGK Deficiency

Phosphorylase Kinase Deficiency of Liver and Muscle*

Prader-Willi Syndrome

Protein C; Thrombophilia V(Protein C Resistance); Thrombosis Risk Factor (Factor V Leiden)

Refsum Syndrome, Adult*; Phytanic Acid Oxidase Deficiency, Adult

Refsum Syndrome, Infantile*; Phytanic Acid Oxidase Deficiency, Infantile

Rh C Genotyping

Rh D Genotyping

Rh E Genotyping

Sex-Determining Region Y*; SRY

Siemerling-Creutzfeldt Disease

Spinal Muscular Atrophy Types I/II/III; Kugelberg-Welander; SMA; Werdnig-Hoffmann Disease

Spinocerebellar Ataxia Type VII*; Olivopontocerebellar Atrophy III; SCA7

Spinocerebellar Ataxia Type VI*; SCA6

Spinocerebellar Ataxia Type I*; Olivopontocerebellar Atrophy I; SCA1

Spinocerebellar Ataxia Type II*; Olivopontocerebellar Atrophy, Holguin; SCA2

Spinocerebellar Ataxia Type III*; Machado-Joseph Disease; SCA3

Spinocerebellar Ataxia Type VII*; SCA8

Tay-Sachs Disease*; GM2 Gangliosidosis

Thanatophoric Dysplasia Type I*

Thanatophoric Dysplasia Type II*; Cloverleaf Skull with Thanatophoric Dysplasia;

Thanatophoric Dysplasia with Kleeblattschaedel

Thrombosis Risk Factor (Factor V Leiden)

Williams Syndrome

X Inactivation Studies

Y Chromosome Detection/Molecular Genetics

Zellweger syndrome*; Cerebrohepatorenal Syndrome

Zygosity Testing*; Twinning

### EXAMPLE8

Assessment of the Current Risk of Cardiovascular Disease

An individual with a family history of cardiovascular disease might be offered a test for variations in the apolipoprotein E (apoE) gene and gene product. Three variant forms of the apoE gene are currently recognized, Apoe2, Apoe3, and Apoe4 reflecting various combinations of variations at two different positions within the gene. Analysis of variations at the ApoE gene can be made either by analysis of lipoproteins present in the blood or by molecular analysis of DNA. Individuals having the Apoe4 form of the gene are at increased risk of cardiovascular disease due to elevated levels of cholesterol and fatty acids. Individual with the apoE4 genotype would be counseled today to implement a diet low in cholesterol and fatty acids and initiate therapy with statins such as Lipitor, Zocor, or Pravachol or other cholesterol lowering agents, and treatment with drugs to control blood pressure such as B-blockers or diuretics. Current practice would involve obtaining consent for a genetic ApoE test (only if the test were performed on DNA) and counseling the individual on the increased risk of cardiovascular disease if they have the ApoE4 variant of this gene

To provide individuals with a current assessment of genetic risk on an ongoing basis, informed consent would be obtained for DNA banking, creating a record with information about their medical concerns, family history, and medical history, and notifying the individual or their healthcare provider when new variances or genes are described in reports of genomic research that would affect their assessment of genetic risk.

Monitoring of reports of genomic research will be performed on an ongoing basis to identify reports of new variances or new gene tests that may be used to refine the assessment of the individuals risk of cardiovascular disease. For example, several clinical trials are currently assessing the potential impact of additional variances within the ApoE gene, particularly variances occurring in the promoter region, which may identify additional haplotypes of the ApoE gene which may be more tightly associated with cardiovascular disease. In addition, validated genetic tests are likely to be developed for other genes which may affect an individuals risk of cardiovascular disease among genes that are homologous to ApoE or share sequence motifs or domains with ApoE, genes on pathways for cholesterol and lipid metabolism, or other genes involved in mediating damage to the vascular endothelium including, but not limited to, factors which regulate growth of endothelium, inflammation, or oxidant damage. In addition, current research suggests that variations in the CETP (cholesterol ester transferase protein) can be used to differentiate those individuals who are likely to respond to Pravacol, and those who are not. Ongoing studies with other cholesterol lowering drugs are likely to refine the ability to select the proper drug for an individual and the dose at which that drug is most likely to be effective (pharmacogenomics). Similarly, tests have been identified for genes that influence the response to B-blockers and diuretics that are commonly used in the prevention or treatment of cardiovascular disease.

When reports of such studies are published and identified by systems for monitoring genomic research, the individual will be contacted using the method authorized by the informed consent. This may involve the individual logging into a secure web site to retrieve personalized information or communication to the individual by email, mail, fax, telephone, or other medium. The individual will be notified of the availability of new genetic tests and offered retesting and recounseling with a current assessment of genetic risk.

### EXAMPLE 8

Assessment of the Current Risk of Cancer

An individual with a family history of breast cancer might be offered a test for variations in the BRCA1 gene. Certain sequence variations within BRCA1 gene are known to be associated with a significantly increased risk of breast cancer. Current practice would involve obtaining consent for a BRCA1 test, counseling the individual on the potential risks and benefits of the test as well as the genetic test results, and implementing screening or prophylactic measures if the test is positive. Individual with mutations known to increase the risk of breast cancer may be counseled to have routine radiological surveillance for early lesions and may even choose to have prophylactic mastectomy. Some individual may choose to take prophylactic therapy with drugs such as tamoxifen.

Genetic testing for BRCA1 does not provide a complete assessment of the risk of breast cancer, even when coupled with family history and clinical exam. For example, variances are frequently found within the gene that may or may not be associated with an increased risk of breast cancer. Moreover, breast cancer is generally acknowledged to be a multifactorial or polygenic disease in which mutations in several genes are required for a cell to become malignant and inheritance of mutations in several genes can increase the risk of disease. This may include genes that share structural or functional similarity with BRCA1, genes on pathways for apoptosis, DNA repair, angiogeneis, inflammation and immune response, breast tissue development, steroid metabolism, and steroid-dependent gene regulation.

To provide individuals with a current assessment of their genetic risk on an ongoing basis, informed consent is obtained for DNA banking, creating a record containing information about the individual's medical concerns, family history, and medical history, and notifying the individual or their healthcare provider when new variances or genes are described in reports of genomic research that would affect assessment of genetic risk.

Monitoring of reports of genomic research are performed on an ongoing basis to identify reports of new variances or new gene tests that may be used to refine the assessment of the individuals risk of cardiovascular disease. For example, several clinical trials are currently assessing the impact of variances within the BRCA1 gene to determine which variances are associated with an increased risk of breast cancer and which are not. In addition, validated genetic tests are likely to be developed for other genes that may contribute to, or protect against, malignancy. Other research is aimed at identifying genes that may predict the efficacy of drugs such as tamoxifen and other chemotherapeutic agents that may be used to prevent or treat breast cancer.

When reports of such studies are published and identified by systems for monitoring genomic research, the individual will be contacted using the method authorized by the informed consent. This may involve the individual logging into a secure web site to retrieve personalized information or communication to the individual by email, mail, fax, telephone, or other medium. The individual will be notified of the availability of new genetic tests and offered retesting and recounseling with a current assessment of genetic risk.

I claim:

1. A method for performing an assessment of genetic risk for an individual concerned about a specific clinical outcome comprising:
obtaining patient consent for genetic testing and assessment of genetic risk for said outcome;
obtaining a DNA sample for DNA testing from said patient;
testing said sample for genes and variations known to be involved in said genetic risk for said outcome;
counseling said patient on test results and assessment of genetic risk;
recording said patient's identity, consent record, contact information, clinical concerns, and genetic test results in a secure and private matter.

2. The method according to claim 1, further comprising:
monitoring genomic research for genes and variations that contribute to said clinical outcome; and
notifying said patient concerning newly discovered genes and variations that contribute to said genetic risk.

3. The method according to claim 2, further comprising:
re-testing said DNA sample for newly discovered genes and variations that contribute to genetic risk; and
re-counseling said patient on said test results and current assessment of genetic risk.

4. The method according to claim 1 wherein said method is performed using a system of networked computers comprising software for organization of database information, secure transactions, web browser readable documents and forms, software for searching online documentation regarding genetic research.

5. The method according to claim 2 wherein said method is performed using a system of networked computers comprising software for organization of database information, secure transactions, web browser readable documents and forms, software for searching online documentation regarding genetic research.

6. The method according to claim 3 wherein said method is performed using a system of networked computers comprising software for organization of database information, secure transactions, web browser readable documents and forms, software for searching online documentation regarding genetic research.

7. The method according to claim 4 wherein said steps are performed in an integrated manner.

8. The method, according to claim 5 wherein said steps are performed in an integrated manner.

9. The method according to claim 6 wherein said steps are performed in an integrated manner.

10. The method according to claim 5 wherein said monitoring is performed in a periodic manner.

11. The method according to claim 5 wherein said monitoring is performed in a continuous manner.

12. A method for performing an assessment of genetic risk for an individual concerned about a specific clinical outcome comprising at least two of the steps of:
obtaining patient consent for genetic testing and assessment of genetic risk for said outcome;
obtaining a DNA sample for DNA testing from said patient;
testing said sample for genes and variations known to be involved in said genetic risk for said outcome;
counseling said patient on test results and assessment of genetic risk;
recording said patient's identity, consent record, contact information, clinical concerns, and genetic test results in a secure and private matter;
monitoring genomic research for genes and variations that contribute to said clinical outcome;
notifying said patient concerning newly discovered genes and variations that contribute to said genetic risk;
re-testing said DNA sample for newly discovered genes and variations that contribute to genetic risk; and
re-counseling said patient on said test results and current assessment of genetic risk.

13. A method for performing an assessment of genetic risk for an individual concerned about a specific clinical outcome comprising the steps of:
obtaining patient consent for genetic testing and assessment of genetic risk for said outcome;
obtaining a DNA sample for DNA testing from said patient;
testing said sample for genes and variations known to be involved in said genetic risk for said outcome;
counseling said patient on test results and assessment of genetic risk;
recording said patient's identity, consent record, contact information, clinical concerns, and genetic test results in a secure and private matter;
monitoring genomic research for genes and variations that contribute to said clinical outcome;
notifying said patient concerning newly discovered genes and variations that contribute to said genetic risk;
re-testing said DNA sample for newly discovered genes and variations that contribute to genetic risk; and
re-counseling said patient on said test results and current assessment of genetic risk, whereby said individual's informed consent is obtained.

14. The method according to claim 13 wherein said steps are performed in an integrated manner.

15. The method according to claim 13 wherein said monitoring is performed in a periodic manner.

16. The method according to claim 13 wherein said recording results in a record comprising said patient's identity, consent record, contact information, clinical concerns and genetic test results that is maintined in a separate environment or medium.

17. The method according to claim 16 wherein said separate environment or medium comprises a secure web site.

18. A system for providing a current assessment of risk for an individual concerned about a specific clinical outcome comprising:
a record including a patient's identity, consent record, contact information, clinical concerns and genetic test results;
a subsystem for monitoring genomic research for genes and variations that contribute to said clinical outcome;
and a subsystem for notifying said patient when the monitoring subsystem identifies new genetic test that may be used for updating said genetic test results.

19. The system according to claim 18 wherein said system comprises a secure web site.
